SEQUENCE LISTING (18 sequences)

SEQ ID NO: 1 - DNA, 3364 bp, Lactuca sativa
  misc_feature 1..3364: note = gDNA LsKO2 WT (Burovia)
  misc_feature 1242: note = /note="n = a or t or c or g"
  misc_feature 1248: note = /note="n = a or c or t or g"
  misc_feature 1344: note = /note="n = a or c or t or g"
  source 1..3364: mol_type = genomic DNA
atggatggcgtgatcgatatgcaaacaattcccctgagaaccgccatagccataggcggcactgccgtcgcacttgtagttgctctttacttctggtttctcagaagctatgcctctccctctcatcacagcaaccatctccctcctgttccaggtactccgattctctgatatttggatttatgtggatgtatgtatgttctactccgttctaactaacaatttgttgaaattcgtattatacaaatgcgaaattgatatgaattttgttggttctttagttgatttgatcttaatacctatctgtctgattgatgatgatgatgaatttgcaagtttgatgacgagtgaaattggaattggaatgtttcagaggtgcctggggtaccagtgttggggaatttactgcagttaaaggagaagaaaccatacatgacttttacaaaatgggcagagatgtatgggcctatttactccattagaactggagccacttccatggtggtggttagctccaatgaaatcgccaaggaggtactcttccccttctctctaatattttaataattaaacaattattagcatgaattacactgtgagagccaggtttaattaataatcaaatgtttaaaacatataaaataagattaagataatagattgttcgtaagatgtgtaatatttattaataaataaatagataattttatattcacttgtattcatagtttattccttgttttttttttaaatgctttttcggtgtaagaacaaatgtactatttgtcttttattggattttggatcgtattacgtgttttacctgttaaaggatctacgtgtaaatttatttatttatacattaataattattaatttaaatgttttttattctggaatgaagatgtataccgtttaatttttttgaagcagttaaccaaaacgtcttttaatcaataataaaatatataagaattcctttattaatacaatattgagaaaatgattctcaatcacggttaattttgcttgctgttatttctcgaagggtaaattggtagattttgtaaaacactagtcccaataagaaataatatatttttttatgccttcaactagataatgttatatttgccgacttcccactgcctttttgcatgtataaaatactttatttaacattttcgaaataatggatgattgttttatataaccattacataattacaaatttagtaaaanaaaatnactttatttaatttatcgttgttgtgtgttacttaatattatatttcaaacgtgtataacgttttaccaatttaaccatgtataaaacccatgtantaaaaactactttatcataaattaaattaatgtaacatactttactctaatgtgtaaatttctatatctgttgcatttaattcaacgatttgattaacatatgactaataaatgcacaatatttggtataggtagttgttacaagatttccatcaatctcaactagaaagctatcatatgcattaaaagtcctgaccgaagataaatccatggttgctatgagtgattacaatgactatcacaaaaccgtaaaacgccatatactcaccgctgtcttgggaccaaatgcccaggttcgtcacattaatttttgcagtatcaacaatcacagtgtcagagtttcaattccaatgtggatttgatttcttcagcacacacacaactgtctgcctgatttacttctcacatgatgatctttggaaacgacagtaacccttttggcctatatggctatattaactacttttttttttttttttttcagaagaaatttcgtgctcatagagacaccatgatggagaatgtttcaaacgaactacatgcgtttttcgaaaaaaatcctaatcaagaagtgaacctcaggaaaatattccaatctcaactttttggactagcaatgaaacaagtgagttatcttaatgcgacaaaaaatacaatgggacaaaagtaaaaaagttgagacatgtctgtctgtaaaaaagacaaaaatacccttctcgtcctcattattgaatgtaaccctaaaaaatctgtctagtatgtcatgtcatgtgtaataattcgagaaaacataaaatatttcatgatacttttacaggcattagggaaggatgttgaaagcatatacgtgaaggatctagaaaccaccatgaaaagagaggaaatctttgaggtattagtggttgacccaatgatgggtgccattgaagttgactggagagatttctttccatatctcaagtgggtcccaaataaaagctttgaaaacataatccatcgaatgtatacccgaagagaagctgtaatgaaagctcttattcaggaacataaaaaacgtattgcatcaggcgaggtaattttgtgtttttatttccatagattatataattcttgctaataataacttttttgttaattaattaaataacagaacttgaacagctacattgattacttgctatcagaagcacaaacattaacagataaacagcttcttatgtctttatgggagccaattattgaatcatcagacactaccatggtcaccacagaatgggcaatgtatgaacttgctaaaaatccaaacatgcaggtttgtttgttttttttttctttaaaatcacatgaattaatgagctaaataatttcaatttgatttttttttttgttaattcattttaggatcgtctatatgaggaaatccaaagtgtttgtggatcagagaagatcacagaagagaatttatctcagcttccatacttatatgctgtttttcaagagaccttgagaaaacattgtcctgttccaataatgcccctgagatacgtccacgagaatacagttctaggaggctaccatgtcccagctggaaccgaggtttttctccttttttttagaaagaaaaaaaaaaaataggttaatatatatagaagggtaaaatggtcatttactgaaattctagtaacaggttgtgaattattttgttgtgtaggtggcgataaatatatatggatgtaacatggataagaaggtgtgggagaatcctgaagagtggaatcctgagaggtttttgagtgaaaaggagagcatggacttgtataaaacaatggcatttgggggaggaaaaagggtgtgtgcaggttctcttcaagccatggtgatttcttgcattggaattggaagattggtgcaggattttgagtggaaactgaaagatgatgctgaagaagatgtgaatacacttgggcttacaacacaaaagcttcatccacttcttgcactcataaaccctagaaaatga

SEQ ID NO: 2 - DNA, 3391 bp, Lactuca sativa
  misc_feature 1..3391: note = gDNA LsKO2 modified
  source 1..3391: mol_type = genomic DNA
atggatggcgtgatcgatatgcaaacaattcccctgagaaccgccatagccataggcggcacggctgtcgcacttgtggttgctctttacttctggtttctcagaagctatgcctctccctctcatcacagcaaccatctccctcctgttccaggtactccgattctctgatatttggatttatgtagatgtatgtatgttctactcggttctaactaacagtttgtggagattcgtattatacaaatgcgaaattgatatgaattttgttggttctttagttgatttgatcttaatacctatctgtctgattgatgatgatgatgaatttgtaagtttgatgacgagtgaaattggaattggaatgtttcagaggtgcctggggtaccagtgttggggaatttactgcagttaaaggagaagaaaccatacatgacttttacaaaatgggcagagatgtatgggcctatttactccattagaactggagccacttccatggtggtggttagctccaatgaaatcgccaaggaggtactcttccccttctctctaatattttaataattaaacaattattagcatgaattagactgtgagagccaggtttatttaataatcaaatgtttaaaacatataaaataagattaagataatagattgttcgtaagatgtgtaatatttattaataaataaatagataattttatattcacttgtattcatagtttattccttgttttttttttaaatgctttttcggtgtaagaacaaatgtactatttgtcttttattggattttggatcgaattacgtgttttccttgttaaaggatctatttgtaaatttatttatttatacattaataattattaatttaaatgtttttttttctgggatgaagatgtacaccgtttaattttattgaagcagttaaccaaaacgtcttttaatcaataataaaatatataagaattcctttattaatacaatattgagaaaatgattctcaatcacggttaattttgcttgctgttatttctcgaagggtaaattggtagattttgtaaaacactagtcccaataagaaataatatatttttttatgccttcaactagataatgttatatttgccgacttcccactgcctttttgcatgtataaaatactttatttaacattttcgaaataatagataattgttttatataaccattacataattacaaatttagtaaaacaaaattactttatataatttattgttgttgtgtgttacttaatattatatttcaaacatgtataatgttttaccgatttaaccatgcataaaacattactacaaaatgtagtaaaaactactttatcataaattaaattaatgtaacatactttactctaatgtgtaaatttatatatctattgcatttaattcaacgatttgattaacatatgactaataaatgcacaatatttggtataggtagttgttacaagatttccatcaatctcaactagaaagctatcatatgcattaaaagtcctgaccgaagataaatccatggttgctatgagtgattacaatgactatcacaaaaccgtaaaacgccatatactcaccgctgtcttggggccaaatgcccaggttcgtcacattaatttttgcagtatcaacaatcacagtgtcagagtttcaattccaatgtcgatttgatttcttcagcacacacacaactgtctacctgatttacttctcacatgatgaccttaggaaacgacagtaacccttttggcctatatggctatattaactactttttttttattttttttcagaagaaattccgtgctcatagagacaccatgatggagaatgtttcaaatgaactacacgcgtttttcgaaaaaaatcctaatcaagaagtgaacctcaggaaaatattccaatctcaactttttggactagcaatgaaacaagtgagttatcttaatgcaacaaaaaatacaatgggacaaaagtaaaaaagttgagacatgtgtgtctgtagaaaagacaaaaatacccttctcgtcctcattattgaatgtaaccctaaaaaatctgtctagtatgtcatgtcatgcgtaacaaacacagtctgaataataattcaagaaaacataaaatattgcatgatacttttacaggcattggggaaggatgttgaaagcatatatgtgaaggatctagaaaccaccatgaaaagagaggaaatctttgaggtattagtggttgacccaatgatgggtgccattgaagttgactggagagatttctttccatatctcaagtgggtcccaaataaaagctttgaaaacataatccatcgaatgtatacccgaagagaagctgtaatgaaagcccttattcaagaacataaaaaacgtatcgcatcaggcgaggtaattttgtgtttttatttccatagattatatcgttcttgctaataataacttttttgttaattaattaaataacagaagttgaacagctacattgattacttgctatcagaagcacaaacattaacagataaacagcttcgtatgtctttatgggagccaattattgaatcatcagacactaccatggtcaccacagaatgggcaatgtatgagcttgcaaaaaatccaaacatgcaggttggtttgttttttttttctttaaaaacacatgaattaatgagctaaataatttcaatttgatttttttttttgttaattcattttaggatcgtctgtatgaggaaatccaaagtgtttgtggatcagagaagatcacagaagacaatttatctcagcttccatacttatatgctgtttttcaagagaccttgagaaaacattgtcctgttccaataatgcccctgagatacgtccacgagaatacagttctaggaggctaccatgtcccagctggaaccgaggtttttctccttttttttagaaagaaaaaaaaaatataggttaatatatatagaagggtaaaatggtcatttactgaaattctagtaacaggttgtgaattattttgttgtgtaggtggcgataaatatatatggatgtaacatggataagaaggtgtgggagaatcctgaagagtggaatcctgagaggtttttgagtgaaaaggagagcatggacttgtataaaacaatggcatttgggggaggaaaaagggtgtgtgcgggttctcttcaagccatggtgatttcatgcattggaattggaagattggtgcaggattttgagtggaaactgaaagatgatgctgaagaagatgtgaatacacttgggcttacaacacaaaagcttcatccacttcttgctctcataaaccctagaaaatga

SEQ ID NO: 3 - protein, 512 aa, Lactuca sativa
  REGION 1..512: note = KO2 WT (Burovia)
MDGVIDMQTIPLRTAIAIGGTAVALVVALYFWFLRSYASPSHHSNHLPPVPEVPGVPVLGNLLQLKEKKPYMTFTKWAEMYGPIYSIRTGATSMVVVSSNEIAKEVVVTRFPSISTRKLSYALKVLTEDKSMVAMSDYNDYHKTVKRHILTAVLGPNAQKKFRAHRDTMMENVSNELHAFFEKNPNQEVNLRKIFQSQLFGLAMKQALGKDVESIYVKDLETTMKREEIFEVLVVDPMMGAIEVDWRDFFPYLKWVPNKSFENIIHRMYTRREAVMKALIQEHKKRIASGENLNSYIDYLLSEAQTLTDKQLLMSLWEPIIESSDTTMVTTEWAMYELAKNPNMQDRLYEEIQSVCGSEKITEENLSQLPYLYAVFQETLRKHCPVPIMPLRYVHENTVLGGYHVPAGTEVAINIYGCNMDKKVWENPEEWNPERFLSEKESMDLYKTMAFGGGKRVCAGSLQAMVISCIGIGRLVQDFEWKLKDDAEEDVNTLGLTTQKLHPLLALINPRK

SEQ ID NO: 4 - protein, 367 aa, Lactuca sativa
  REGION 1..367: note = KO2 modified
MDGVIDMQTIPLRTAIAIGGTAVALVVALYFWFLRSYASPSHHSNHLPPVPEVPGVPVLGNLLQLKEKKPYMTFTKWAEMYGPIYSIRTGATSMVVVSSNEIAKEVVVTRFPSISTRKLSYALKVLTEDKSMVAMSDYNDYHKTVKRHILTAVLGPNAQKKFRAHRDTMMENVSNELHAFFEKNPNQEVNLRKIFQSQLFGLAMKQALGKDVESIYVKDLETTMKREEIFEVLVVDPMMGAIEVDWRDFFPYLKWVPNKSFENIIHRMYTRREAVMKALIQEHKKRIASGENLNSYIDYLLSEAQTLTDKQLLMSLWEPIIESSDTTMVTTEWAMYELAKNPNMQDRLYEEIQSVCGSEKITEENLS

SEQ ID NO: 5 - DNA, 1417 bp, Lactuca sativa
  misc_feature 1..1417: note = Ls20ox1-B WT (Burovia)
  source 1..1417: mol_type = genomic DNA
atgtcttccctcaaggaagagctcaaagaagaccaacaaaggtcactcgtgttcgatgcatcagtcctacaacacgaaaccaacatcccccaacagttcatatggcccgataatgagaaacccaactccaaaaaatctaaggacctagaggttcctttgatagacttgggaggcttcctttctggacgctcgagttcaacaaaagaagcctccaaactcgttggtaacgcttgccaaaaacatgggtttttcctcgtggtaaatcatggagttgatgcaaacctaatatctgatgctcaaacatacatggacttgttctttgagcttcccctttcagaaaagcaaagagctcaaagaaaagccggtgaaagctgtggatatgctagtagcttcactggaagattctcctctaaacttccttggaaagagacgctatctttccgattctcagccgagaagaactcggccgatatagttaaggactacttcgagaacacaatgggcgaagaatttgttcgactcgggtatgtttcaaatcacacatgtatctagctatctcttttggtaattatataagctagtttttacaaccatagactaaatgatttgattgttgcaggaaggtgtatcaagaatattgcaatgccatgagtagactttctcttgggatcatggagttattggggttgagccttggggttaaccgatctcacttcaaggaatttttcgaagaaaataattctataatgagactcaactactatccacgatgccaaaaaccagagctcaccttaggaacggggcctcattgtgacccgacatcgttgacaattctacaccaagataacgttggtgggctagaagtgttcgtggacaatgagtggcgttccattacaccaaattcaaatgcttttgttgtcaatatcggtgacacattcatggtaatcaacatgttcatgattagcttacatgtaaatggttttacagatagtacagggatggagcctaattggattcttttttccatcttgtactgttttgtttgtatttggtgttttggcttttgtccaagtactttttaactgcattttcttctttttttttcgaagttggttttaatgttactctgtttatttgtttactacaggcactttcaaatggaagatacaaaagttgcttacatagagctgtggtaaacaacaaaactccaagaaagtcacttgctttctttctatgcccaaagaaggataaggtggtgagcccaccaaaggaattggtggatgaaaataacccaagggtatatccagattttacatgggctacttttcttgagttcactcagaagcattacagagctgacatgaacaccctccaagctttttcaaactgggttgaacagaaaaccagcacaacttga

SEQ ID NO: 6 - DNA, 1417 bp, Lactuca sativa
  misc_feature 1..1417: note = Ls20ox1-B modified
  source 1..1417: mol_type = genomic DNA
atgtcttccctcaaggaagatctcaaagaagaccaacaaaggtcactcgtgttcgatgcatcagtcctacaacacgaaaccaacatcccccaacagttcatatggcccgataatgagaaacccaactccaaaaaatctaaggaccttgaggttcctttgatagacttgggaggcttcctttctggacgctcgagttcaacaaaagaagcctccaaactcgttggtaacgcttgccaaaaacatgggtttttcctcgtggtaaatcatggagttgatgcaaacctaatatctgatgctcaaacatacatggacttgttctttgagcttcccctttcagaaaagcaaagagctcaaagaaaagccggtgaaagctgtggatatgctagtagcttcactggaagattctcctctaaacttccttggaaagagacgctatctttccgattctcagccgagaagaactcggccgatatagttaaggactacttcgagaacacaatgggcgaagaatttgttcgactcgggtatgtttcaaatcacacatgtatctagctatctcttttggtatttatataagctagtttttacaaccatagactaaatgatttgattgttgcaggaaggtgtatcaagaatattgcaatgccatgagtagactttctcttgggatcatggagttattggggttgagccttggggttaaccgatctcacttcaaggaatttttcgaagaaaataattctataatgagactcaactactatccacgatgccaaaaaccagagctcaccttaggaacggggcctcattgtgacccgacatcgttgacaattctacaccaagataacgttggtgggctagaagtgttcgtggacaatgagtggcgttccattacaccaaattcaaatgcttttgttgtcaatatcggtgacacattcatggtaatcaacatgttcatgattagcttacatgtaaatggttttacagatagtacagggatggagcctaattggattcttttttccatcttgtactgttttgtttgtatttggtgttttggcttttgtccaagtactttttaactgcattttcttcttcttttttcgaagttggttttaatgttactctgtttatttgtttactacaggcactttcaaatggaagatacaaaagttgcttacatagagctgtggtaaacaacaaaactccaagaaagtcacttgctttctttctatgcccaaagaaggataaggtggtgagcccaccaaaggaattggtggatgaaaataacccaagggtatatccagattttacatgggctacttttcttgagttcactcagaagcattacagagctgacatgaacaccctccaagctttttcaaactgggttgaacagaaaaccagcacaacttga

SEQ ID NO: 7 - protein, 383 aa, Lactuca sativa
  REGION 1..383: note = GA20ox1-B WT (Burovia)
MAIDCMIKTPSCMSSLKEDLKEDQQRSLVFDASVLQHETNIPQQFIWPDNEKPNSKKSKDLEVPLIDLGGFLSGRSSSTKEASKLVGNACQKHGFFLVVNHGVDANLISDAQTYMDLFFELPLSEKQRAQRKAGESCGYASSFTGRFSSKLPWKETLSFRFSAEKNSADIVKDYFENTMGEEFVRLGKVYQEYCNAMSRLSLGIMELLGLSLGVNRSHFKEFFEENNSIMRLNYYPRCQKPELTLGTGPHCDPTSLTILHQDNVGGLEVFVDNEWRSITPNSNAFVVNIGDTFMALSNGRYKSCLHRAVVNNKTPRKSLAFFLCPKKDKVVSPPKELVDENNPRVYPDFTWATFLEFTQKHYRADMNTLQAFSNWVEQKTSTT

SEQ ID NO: 8 - protein, 383 aa, Lactuca sativa
  REGION 1..383: note = GA20ox1-B modified
MAIDCMIKTPSCMSSLKEDLKEDQQRSLVFDASVLQHETNIPQQFIWPDNEKPNSKKSKDLEVPLIDLGGFLSGRSSSTKEASKLVGNACQKHGFFLVVNHGVDANLISDAQTYMDLFFELPLSEKQRAQRKAGESCGYASSFTGRFSSKLPWKETLSFRFSAEKNSADIVKDYFENTMGEEFVRLGKVYQEYCNAMSRLSLGIMELLGLSLGVNRSHFKEFFEENNSIMRLNYYPRCQKPELTLGTGPHCDPTSLTILHQDNVGGLKVFVDNEWRSITPNSNAFVVNIGDTFMALSNGRYKSCLHRAVVNNKTPRKSLAFFLCPKKDKVVSPPKELVDENNPRVYPDFTWATFLEFTQKHYRADMNTLQAFSNWVEQKTSTT

SEQ ID NO: 9 - DNA, 1539 bp, Lactuca sativa
  misc_feature 1..1539: note = CDS LsKO2 WT (Burovia)
  source 1..1539: mol_type = genomic DNA
atggatggcgtgatcgatatgcaaacaattcccctgagaaccgccatagccataggcggcactgccgtcgcacttgtagttgctctttacttctggtttctcagaagctatgcctctccctctcatcacagcaaccatctccctcctgttccagaggtgcctggggtaccagtgttggggaatttactgcagttaaaggagaagaaaccatacatgacttttacaaaatgggcagagatgtatgggcctatttactccattagaactggagccacttccatggtggtggttagctccaatgaaatcgccaaggaggtagttgttacaagatttccatcaatctcaactagaaagctatcatatgcattaaaagtcctgaccgaagataaatccatggttgctatgagtgattacaatgactatcacaaaaccgtaaaacgccatatactcaccgctgtcttgggaccaaatgcccagaagaaatttcgtgctcatagagacaccatgatggagaatgtttcaaacgaactacatgcgtttttcgaaaaaaatcctaatcaagaagtgaacctcaggaaaatattccaatctcaactttttggactagcaatgaaacaagcattagggaaggatgttgaaagcatatacgtgaaggatctagaaaccaccatgaaaagagaggaaatctttgaggtattagtggttgacccaatgatgggtgccattgaagttgactggagagatttctttccatatctcaagtgggtcccaaataaaagctttgaaaacataatccatcgaatgtatacccgaagagaagctgtaatgaaagctcttattcaggaacataaaaaacgtattgcatcaggcgagaacttgaacagctacattgattacttgctatcagaagcacaaacattaacagataaacagcttcttatgtctttatgggagccaattattgaatcatcagacactaccatggtcaccacagaatgggcaatgtatgaacttgctaaaaatccaaacatgcaggatcgtctatatgaggaaatccaaagtgtttgtggatcagagaagatcacagaagagaatttatctcagcttccatacttatatgctgtttttcaagagaccttgagaaaacattgtcctgttccaataatgcccctgagatacgtccacgagaatacagttctaggaggctaccatgtcccagctggaaccgaggtggcgataaatatatatggatgtaacatggataagaaggtgtgggagaatcctgaagagtggaatcctgagaggtttttgagtgaaaaggagagcatggacttgtataaaacaatggcatttgggggaggaaaaagggtgtgtgcaggttctcttcaagccatggtgatttcttgcattggaattggaagattggtgcaggattttgagtggaaactgaaagatgatgctgaagaagatgtgaatacacttgggcttacaacacaaaagcttcatccacttcttgcactcataaaccctagaaaatga

SEQ ID NO: 10 - DNA, 1104 bp, Lactuca sativa
  misc_feature 1..1104: note = CDS LsKO2 (modified)
  source 1..1104: mol_type = genomic DNA
atggatggcgtgatcgatatgcaaacaattcccctgagaaccgccatagccataggcggcactgccgtcgcacttgtagttgctctttacttctggtttctcagaagctatgcctctccctctcatcacagcaaccatctccctcctgttccagaggtgcctggggtaccagtgttggggaatttactgcagttaaaggagaagaaaccatacatgacttttacaaaatgggcagagatgtatgggcctatttactccattagaactggagccacttccatggtggtggttagctccaatgaaatcgccaaggaggtagttgttacaagatttccatcaatctcaactagaaagctatcatatgcattaaaagtcctgaccgaagataaatccatggttgctatgagtgattacaatgactatcacaaaaccgtaaaacgccatatactcaccgctgtcttgggaccaaatgcccagaagaaatttcgtgctcatagagacaccatgatggagaatgtttcaaacgaactacatgcgtttttcgaaaaaaatcctaatcaagaagtgaacctcaggaaaatattccaatctcaactttttggactagcaatgaaacaagcattagggaaggatgttgaaagcatatacgtgaaggatctagaaaccaccatgaaaagagaggaaatctttgaggtattagtggttgacccaatgatgggtgccattgaagttgactggagagatttctttccatatctcaagtgggtcccaaataaaagctttgaaaacataatccatcgaatgtatacccgaagagaagctgtaatgaaagctcttattcaggaacataaaaaacgtattgcatcaggcgagaacttgaacagctacattgattacttgctatcagaagcacaaacattaacagataaacagcttcttatgtctttatgggagccaattattgaatcatcagacactaccatggtcaccacagaatgggcaatgtatgaacttgctaaaaatccaaacatgcaggatcgtctatatgaggaaatccaaagtgtttgtggatcagagaagatcacagaagagaatttatcttag

SEQ ID NO: 11 - DNA, 1116 bp, Lactuca sativa
  misc_feature 1..1116: note = CDS Ls20ox1-B WT (Burovia)
  source 1..1116: mol_type = genomic DNA
atgtcttccctcaaggaagagctcaaagaagaccaacaaaggtcactcgtgttcgatgcatcagtcctacaacacgaaaccaacatcccccaacagttcatatggcccgataatgagaaacccaactccaaaaaatctaaggacctagaggttcctttgatagacttgggaggcttcctttctggacgctcgagttcaacaaaagaagcctccaaactcgttggtaacgcttgccaaaaacatgggtttttcctcgtggtaaatcatggagttgatgcaaacctaatatctgatgctcaaacatacatggacttgttctttgagcttcccctttcagaaaagcaaagagctcaaagaaaagccggtgaaagctgtggatatgctagtagcttcactggaagattctcctctaaacttccttggaaagagacgctatctttccgattctcagccgagaagaactcggccgatatagttaaggactacttcgagaacacaatgggcgaagaatttgttcgactcgggaaggtgtatcaagaatattgcaatgccatgagtagactttctcttgggatcatggagttattggggttgagccttggggttaaccgatctcacttcaaggaatttttcgaagaaaataattctataatgagactcaactactatccacgatgccaaaaaccagagctcaccttaggaacggggcctcattgtgacccgacatcgttgacaattctacaccaagataacgttggtgggctagaagtgttcgtggacaatgagtggcgttccattacaccaaattcaaatgcttttgttgtcaatatcggtgacacattcatggcactttcaaatggaagatacaaaagttgcttacatagagctgtggtaaacaacaaaactccaagaaagtcacttgctttctttctatgcccaaagaaggataaggtggtgagcccaccaaaggaattggtggatgaaaataacccaagggtatatccagattttacatgggctacttttcttgagttcactcagaagcattacagagctgacatgaacaccctccaagctttttcaaactgggttgaacagaaaaccagcacaacttga

SEQ ID NO: 12 - DNA, 1116 bp, Lactuca sativa
  misc_feature 1..1116: note = CDS Ls20ox1-B (modified)
  source 1..1116: mol_type = genomic DNA
atgtcttccctcaaggaagagctcaaagaagaccaacaaaggtcactcgtgttcgatgcatcagtcctacaacacgaaaccaacatcccccaacagttcatatggcccgataatgagaaacccaactccaaaaaatctaaggacctagaggttcctttgatagacttgggaggcttcctttctggacgctcgagttcaacaaaagaagcctccaaactcgttggtaacgcttgccaaaaacatgggtttttcctcgtggtaaatcatggagttgatgcaaacctaatatctgatgctcaaacatacatggacttgttctttgagcttcccctttcagaaaagcaaagagctcaaagaaaagccggtgaaagctgtggatatgctagtagcttcactggaagattctcctctaaacttccttggaaagagacgctatctttccgattctcagccgagaagaactcggccgatatagttaaggactacttcgagaacacaatgggcgaagaatttgttcgactcgggaaggtgtatcaagaatattgcaatgccatgagtagactttctcttgggatcatggagttattggggttgagccttggggttaaccgatctcacttcaaggaatttttcgaagaaaataattctataatgagactcaactactatccacgatgccaaaaaccagagctcaccttaggaacggggcctcattgtgacccgacatcgttgacaattctacaccaagataacgttggtgggctaaaagtgttcgtggacaatgagtggcgttccattacaccaaattcaaatgcttttgttgtcaatatcggtgacacattcatggcactttcaaatggaagatacaaaagttgcttacatagagctgtggtaaacaacaaaactccaagaaagtcacttgctttctttctatgcccaaagaaggataaggtggtgagcccaccaaaggaattggtggatgaaaataacccaagggtatatccagattttacatgggctacttttcttgagttcactcagaagcattacagagctgacatgaacaccctccaagctttttcaaactgggttgaacagaaaaccagcacaacttga

SEQ ID NO: 13 - DNA, 49 bp, Lactuca sativa
  misc_feature 1..49: note = Marker LS06200_A1
  source 1..49: mol_type = genomic DNA
gaaggtgaccaagttcatgctgaaaaacagcatataagtatggaagctg

SEQ ID NO: 14 - DNA, 49 bp, Lactuca sativa
  misc_feature 1..49: note = Marker LS06200_A2
  source 1..49: mol_type = genomic DNA
gaaggtcggagtcaacggattgaaaaacagcatataagtatggaagcta

SEQ ID NO: 15 - DNA, 29 bp, Lactuca sativa
  misc_feature 1..29: note = LS06200_C1
  source 1..29: mol_type = genomic DNA
atccaaagtgtttgtggatcagagaagat

SEQ ID NO: 16 - DNA, 44 bp, Lactuca sativa
  misc_feature 1..44: note = Marker LS06187_A1
  source 1..44: mol_type = genomic DNA
gaaggtgaccaagttcatgctcactcattgtccacgaacacttc

SEQ ID NO: 17 - DNA, 45 bp, Lactuca sativa
  misc_feature 1..45: note = Marker LS06187_A2
  source 1..45: mol_type = genomic DNA
gaaggtcggagtcaacggattccactcattgtccacgaacacttt

SEQ ID NO: 18 - DNA, 29 bp, Lactuca sativa
  misc_feature 1..29: note = Marker LS06187_C1
  source 1..29: mol_type = genomic DNA
cgttgacaattctacaccaagataacgtt

(19) United States
(12) Patent Application Publication
(10) Pub. No.: US 2026/0132414 A9
(43) Pub. Date: May 14, 2026

(54) SHADE TOLERANT LETTUCE

(71) Applicant: RIJK ZWAAN ZAADTEELT EN ZAADHANDEL B.V., De Lier (NL)

(72) Inventors: Carolina Myluska CARO RIOS, De Lier (NL); Cornelis Maria Petrus VAN DUN, De Lier (NL)

(21) Appl. No.: 18/493,025

(22) Filed: Oct. 24, 2023

Related U.S. Application Data
(63) Continuation-in-part of application No. PCT/EP2022/062211, filed on May 5, 2022.

(30) Foreign Application Priority Data
May 5, 2021 (WO) PCT/EP2021/061912

(51) Int. Cl. C12N 15/82; A01H 1/04

(52) U.S. Cl. CPC C12N 15/8271; A01H 1/045; C12N 15/821

(57) ABSTRACT

The present invention relates to a lettuce plant, with a modified LsKO2 and/or Ls20ox1-B gene, wherein the homozygous presence of said genes leads to a shade tolerant phenotype, the wild-type LsKO2 gene and the wild-type Ls20ox1-B gene can have a coding sequence having at least 70% sequence identity to SEQ ID NO.: 9 and SEQ ID NO.: 11, respectively. The modification involves replacement and/or deletion and/or insertion of nucleotides resulting in an absence of functional KO2 and/or GA20ox1-B protein or the modification results in the absence of the wild-type LsKO2 and/or Ls20ox1-B gene. The homozygous presence of one or both of the modified LsKO2 and Ls20ox1-B genes or the homozygous absence of one or both of the wild-type LsKO2 and/or Ls20ox1-B genes in the plant confers shade tolerance as compared to a plant with the wild-type LsKO2 and Ls20ox1-B gene and not showing shade tolerance.

### RELATED APPLICATIONS AND INCORPORATION BY REFERENCE

This application is a continuation-in-part application of international patent application Serial No. PCT/EP2022/062211 filed 5 May 2022, which published as PCT Publication No. WO 2022/234045 on 10 Nov. 2022, which claims benefit of international patent application Serial No. PCT/EP2021/061912 filed 5 May 2021.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing which has been submitted electronically and is hereby incorporated by reference in its entirety. Said XML copy, was created on Oct. 4, 2023, is named Y7954-00584SL.xml, and is 40,941 bytes in size.

### FIELD OF THE INVENTION

The present invention relates to a shade tolerant lettuce plant. More specifically, the present invention relates to a lettuce plant that comprises one or more modified gene homolog(s) that impart shade tolerance to the plant. The invention further relates to the modified gene homolog(s) and a method for obtaining a lettuce plant with enhanced shade tolerance as well as to methods for identifying and selecting a lettuce plant having enhanced shade tolerance and to plant parts, progeny, seed and fruit of the plant having enhanced shade tolerance.

### BACKGROUND OF THE INVENTION

Light is one of the most important environmental factors affecting plant growth and development. A decrease in the quantity and change in the quality of light, as a result of shading by neighboring plants, triggers a suite of responses termed the “shade avoidance syndrome” (SAS). SAS is a complex set of growth and developmental responses that aims to prevent or counteract the effects of shading. Although SAS is an adaptive response in a natural setting, it is highly undesired in plant cultures, especially in case of dense stands or competing weeds, as it adversely affects the yield of harvestable components.

Shade is predominantly experienced by the plant as a decrease in the red/far-red (R/FR) ratio of incoming light. A low R/FR ratio acts as a shade signal to trigger the SAS. Interactions between photoreceptors and phytochrome-interacting factors activate downstream signaling pathways, which in turn activate transcription factors and lead to changes in the concentrations of various phytohormones, ultimately leading to adaptive changes in the morphology and physiology of the plant. Among the phytohormones, especially auxin, gibberellins, ethylene and brassinosteroids are involved in the SAS processes.

Responses to shade are varied; they predominantly affect the vegetative part (elongation of internodes, hypocotyl and petioles; decreasing leaf expansion; reduction of branching; reduced root growth), however, prolonged shade leads to changes in the reproductive organs as well, leading to accelerated flowering, reduced seed set, and reduced germination capacity. In addition, the SAS influences the overall fitness of the plant; for example, it increases the plant's susceptibility towards pathogens.

The shade avoidance syndrome is a major hindrance to indoor farming technologies and hydroponic culturing. Indoor farming (also termed controlled-environment agriculture) is the practice of growing plants entirely indoors. One form of indoor farming is vertical farming, where plants are grown in vertically stacked layers. Indoor farming often incorporates hydroponic culturing or hydroponics, a soil-less culturing technique in which plants are grown directly in nutrient solutions. The various forms of indoor farming technologies present themselves as attractive solutions to the growing demand for close-to-source produce. In addition to reducing transportation costs and time, and hence the carbon footprint, they drastically reduce the growth space that is needed and provide a highly standardized, disease-free and controlled growth environment, resulting in exceptionally high crop yields.

Indoor growing technologies are based on artificial lighting systems and high density planting. Under these circumstances the SAS is triggered in most crop species. Furthermore, application of far-red enriched light recipes in such high-tech environments to increase productivity is severely limited by the negative effects of the SAS on quality. In addition, indoor growing technologies are closed production systems, and as such, prone to high internal heat loads. Elevated temperatures can trigger a so-called thermomorphogenesis response in the plants, which adds to the SAS by further enhancing the elongation of the vegetative part, thereby lowering quality. Therefore, application of a moderately elevated ambient temperature to enhance productivity is severely limited by this developmental response.

Leafy vegetables are vegetables whose harvestable product is the green leaf, consumed fresh or cooked. Lettuce is among the most important leafy vegetables worldwide. Lettuce comprises mainly the various cultivars of Lactuca sativa. Lactuca sativa belongs to the Cichoreae tribe of the Asteraceae (Compositae) family. Lettuce is related to chicory, sunflower, aster, scorzonera, dandelion, artichoke and chrysanthemum. Lactuca sativa is one of about 300 species in the genus Lactuca.

As a disease-free growing method, the indoor farming technology is particularly interesting for the production of lettuce, as it provides a vegetable product which is completely free of pesticide residues and/or parasitic contamination. However, indoor growing technologies create shading conditions and hence trigger the shade avoidance syndrome, which greatly decreases the crop's quality and marketability.

In lettuce, the shade avoidance syndrome of the vegetative part is characterized by the elongation of the internodes, petiole elongation, hyponasty, narrowing and bleaching of the leaf lamina, and reduced root development. In relation to the reduced root development, tip-burn can occur.

In the current practice, the shade avoidance syndrome is attenuated by adjusting the growing conditions (e.g. light spectrum, temperature) but this solution offers only a partial result and decreases the plant's productivity.

Citation or identification of any document in this application is not an admission that such document is available as prior art to the present invention.

### SUMMARY OF THE INVENTION

There is a need in the art to provide lettuce plants with reduced shade avoidance syndrome. It is therefore an object of the present invention to address the above need in the art and tackle the problem of shade avoidance in a fundamental manner, by finding a genetic solution according to which plants inherently show a reduced shade avoidance syndrome.

In the research that led to the present invention mutant lettuce plants were isolated that showed a reduced shade avoidance syndrome. It was surprisingly found that mutations in the ent-kaurene oxidase-2 (LsKO2) gene that reduce the level or activity of the KO2 protein or lead to a complete absence of the protein, cause the mutant plant to have a reduced shade avoidance syndrome as compared to the same plant not having such a mutation, provided that the mutant LsKO2 gene is homozygously present in the plant.

Furthermore, it was surprisingly found that mutations in the gibberellin-20-oxidase-1-B (Ls20ox1-B) gene that reduce the level or activity of the GA20ox1-B protein or lead to a complete absence of the protein, also cause the mutant plant to have a reduced shade avoidance syndrome as compared to the same plant not having such a mutation, provided that the mutant Ls20ox1-B gene is homozygously present in the plant.

The proteins ent-Kaurene Oxidase 2 (KO2) and gibberellin-20-oxidase 1-B (GA20ox1-B) are key enzymes of the gibberellin biosynthesis pathway. KO2 belongs to the P-450 monooxygenase family and catalyses three successive oxidations of the 4-methyl group of ent-kaurene giving kaurenoic acid. GA20ox1-B is a multifunctional enzyme that catalyzes the sequential oxidation of GA12 and GA53 to GA9 and GA20 respectively, leading to bioactive gibberellins.

Gibberellins are plant hormones which play important roles in plant development, controlling the processes of seed germination, cell elongation, flowering, embryogenesis and seed development. Control of such processes is achieved by the development-dependent and organ-specific adjustment of the concentrations of biologically active gibberellins.

Fundamental research in model plant species, such as Arabidopsis has shown that many phytohormones are involved in the shade avoidance syndrome, with the hormone auxin acting as key regulator.

Furthermore, when the biosynthesis of bioactive gibberellic acids is impaired, growth is restrained, irrespective of shading conditions. Thus, it was surprising to find that the above described mutations of the gibberellin biosynthesis gene LsKO2 and/or gibberellin biosynthesis gene Ls20ox1-B in lettuce do not lead to reduced plant height and reduced stem internode length, generally associated with a decreased expression of the gibberellic acid biosynthesis enzymes, when shading conditions are not present.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### Deposit

Seeds of lettuce (Lactuca sativa) with a mutated ent-kaurene oxidase 2 (LsKO2) gene or a mutated gibberellin-20-oxidase 1-B gene (Ls20ox1-B) were deposited with NCIMB Ltd, Ferguson Building, Craibstone Estate, Bucksburn, Aberdeen AB21 9YA, UK on 10 Jan. 2020 under accession number NCIMB 43547 and under accession number NCIMB 43548, respectively. were made and accepted pursuant to the terms of the Budapest Treaty. Upon issuance of a patent, all restrictions upon the deposit will be removed, and the deposit is intended to meet the requirements of 37 CFR §§ 1.801-1.809. The deposit will be irrevocably and without restriction or condition released to the public upon the issuance of a patent and for the enforceable life of the patent. The deposit will be maintained in the depository for a period of 30 years, or 5 years after the last request, or for the effective life of the patent, whichever is longer, and will be replaced if necessary during that period.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, given by way of example, but not intended to limit the invention solely to the specific embodiments described, may best be understood in conjunction with the accompanying drawings.

FIG. 1. Lettuce plants growing in a plant factory (left) and one typical example of the undesired shade avoidance phenotype (right).

FIG. 2. Setup of the pilot experiment, with white plastic (left, control) and green plastic (left, test condition). Below the pictures, the light spectrum beneath the plastic is shown.

FIG. 3. Characteristics of the typical shade avoidance syndrome phenotype in young lettuce plant.

FIG. 4. Young plants of Lactuca sativa ‘Burovia’ showing the typical shade avoidance syndrome and mutant plant.

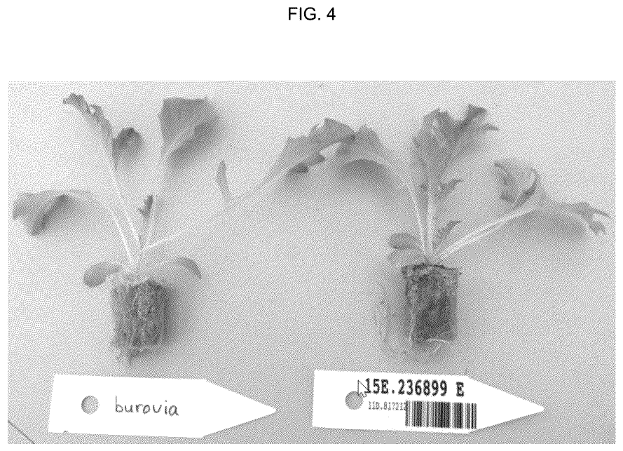

FIG. 5. Experimental setup of the genetic screen for elongation mutants.

FIG. 6. Comparison of a lettuce head of a shade tolerant mutant line and that of the non-mutated parent (Burovia 8405RZ). The plants were grown in a greenhouse under the conditions defined herein, and harvested when fully grown, just before complete maturity.

FIG. 7. Comparison of the ratio of red light to far-red light (R/FR ratio) under the different light filters.

FIG. 8. The phenotypic analysis of shoot elongation responses, based on the length of the hypocotyl.

FIG. 9. The phenotypic analysis of shoot and root elongation responses.

FIG. 10. Pit length (cm) and weight (g) of fully grown lettuce heads of selected shade tolerant mutant lines compared to the non-mutant parent (Burovia RZ).

FIG. 11. Nucleotide sequences of the genetic marker used to identify a mutation at position 200841726 on TgG6 (gene LsKO2).

FIG. 12. Nucleotide sequences of the genetic marker used to identify a mutation at position 72575669 on IgG9 (gene Ls20ox1-B).

FIG. 13. Sequence identity corresponding to the sequences claimed in the present invention and their wild-type origin. Abbreviations: gDNA=genomic DNA, CDS=coding sequence, WT=wild-type.

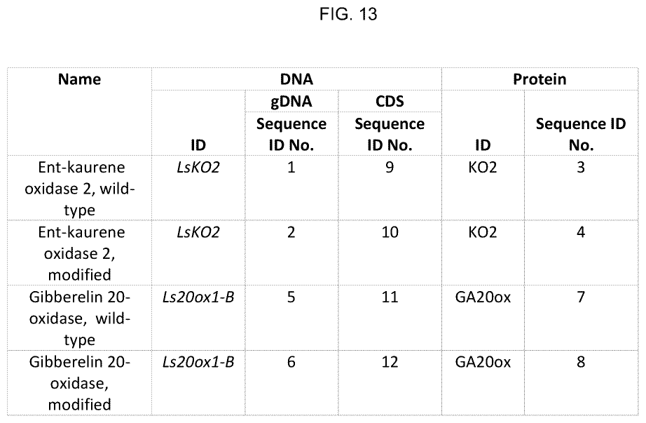

FIG. 14. Genomic DNA sequence of wild type LsKO2 (SEQ ID NO.: 1).

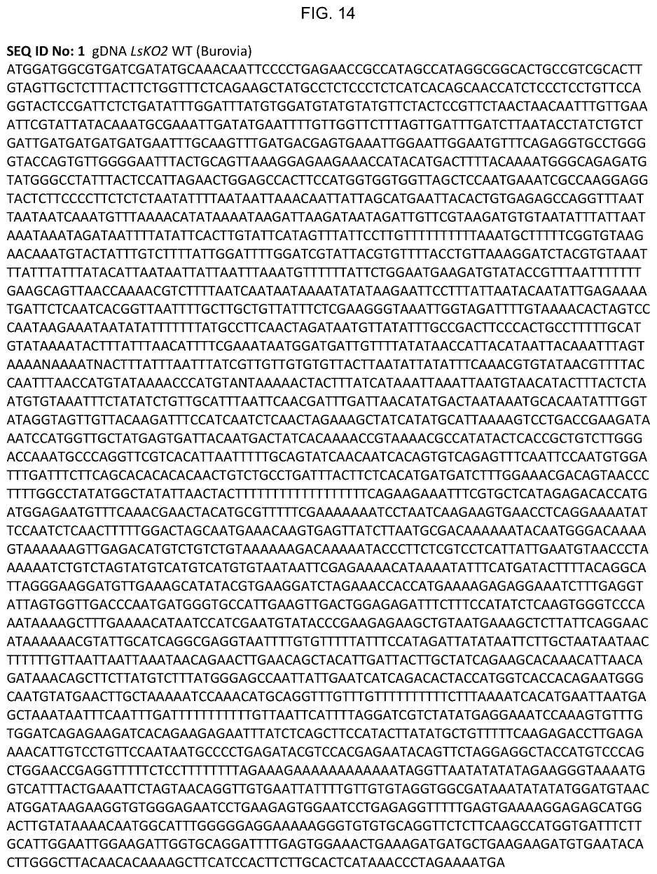

FIG. 15. Genomic DNA sequence of modified LsKO2 ene (SEQ ID NO.: 2).

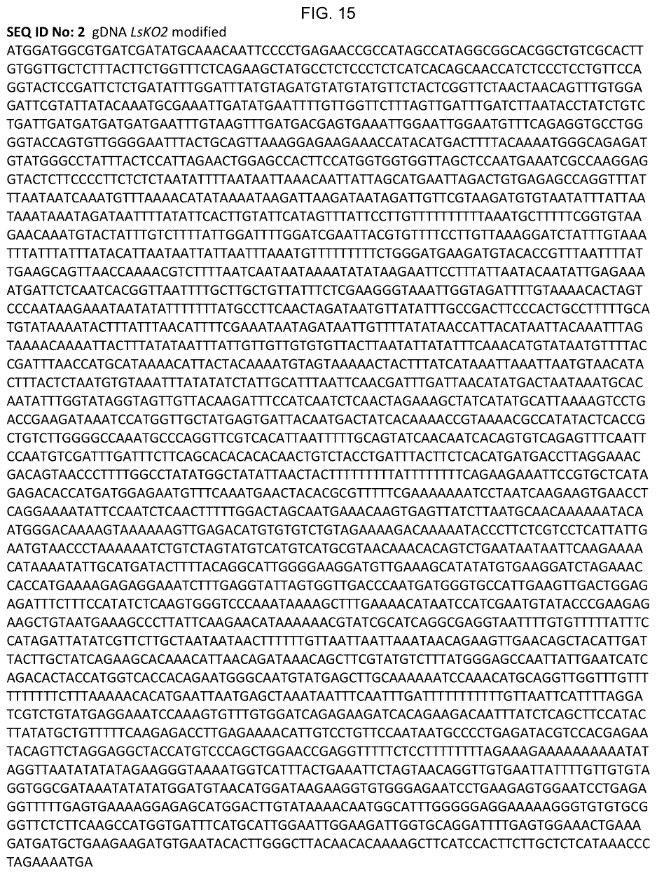

FIG. 16. KO2 wild-type (Burovia) sequence (SEQ ID NO.: 3).

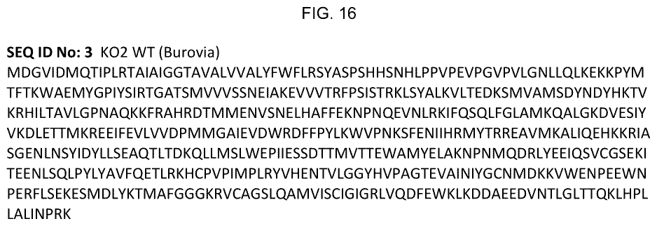

FIG. 17. KO2 modified sequence (SEQ ID NO.: 4). The premature stop codon is marked with an asterisk (*).

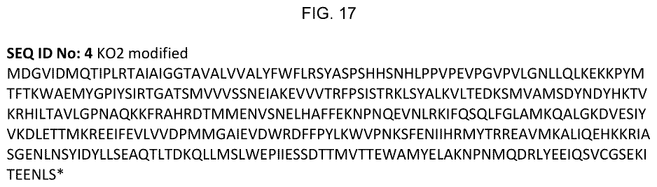

FIG. 18. Ls20ox1-B wild-type (Burovia) sequence (SEQ ID NO.: 5).

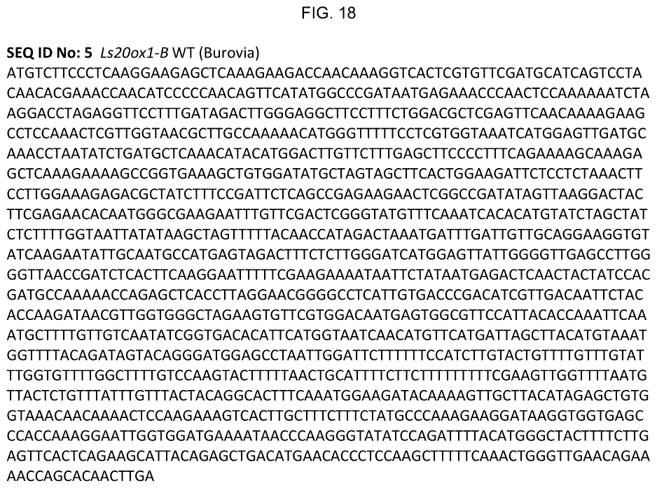

FIG. 19. Ls20ox1-B modified sequence (SEQ ID NO.: 6).

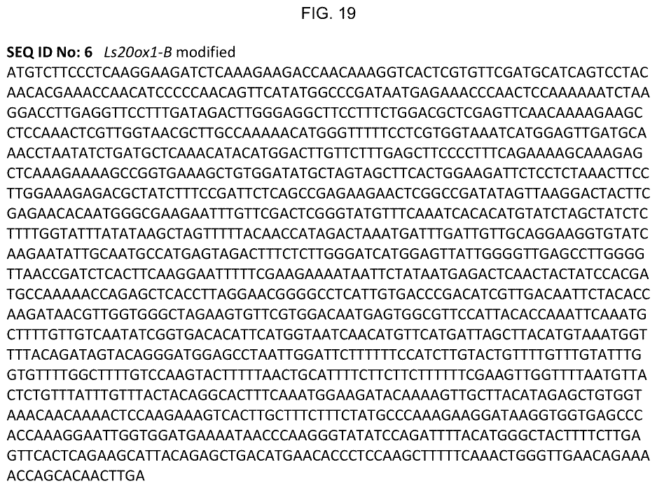

FIG. 20. GA20ox1-B wild-type (Burovia) sequence (SEQ ID NO.: 7). The premature stop codon is marked with an asterisk (*).

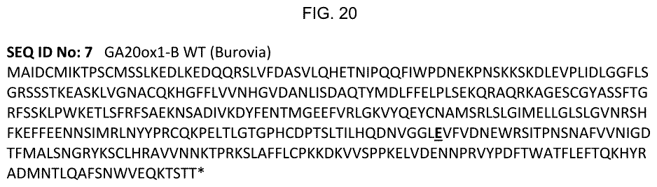

FIG. 21. GA20ox1-B modified sequence (SEQ ID NO.: 8). The premature stop codon is marked with an asterisk (*). The substitution of the present invention is marked in bold and underlined.

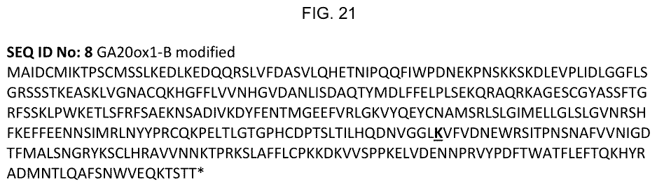

FIG. 22. Coding sequence of wild-type LsKO2 (Burovia) (SEQ ID NO.: 9).

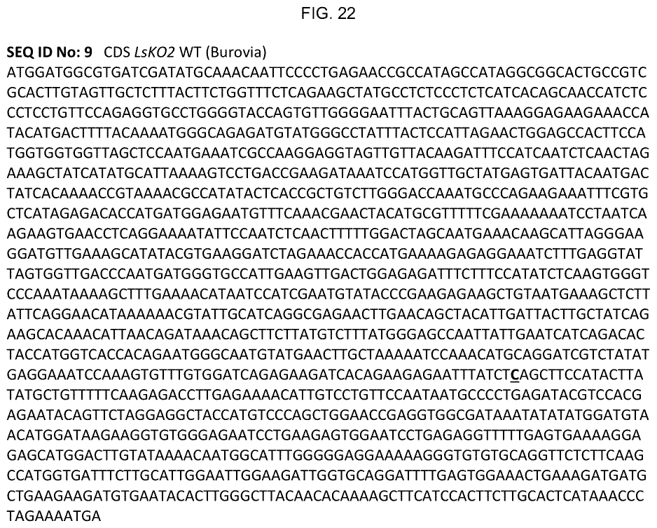

FIG. 23. Coding sequence of modified LsKO2 (SEQ ID NO.: 10). The premature stop codon is marked with an asterisk (*). The substitution of the present invention is marked in bold and underlined.

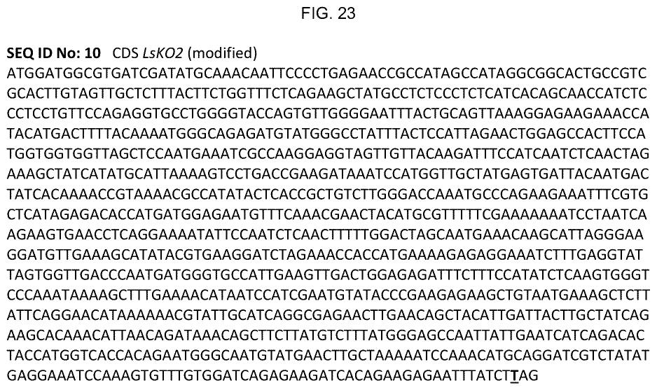

FIG. 24. Coding sequence of wild-type Ls20ox1-B (Burovia) (SEQ ID NO.: 11).

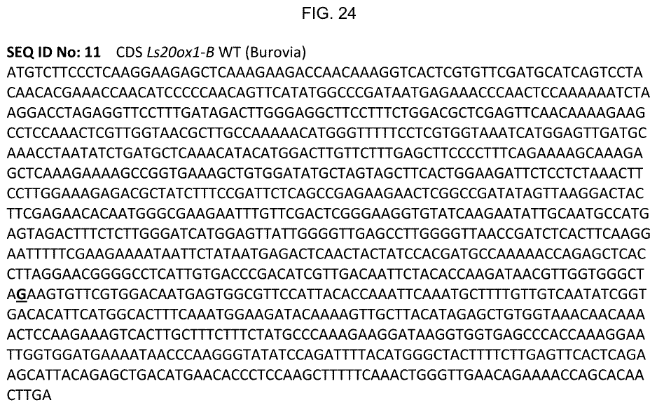

FIG. 25. Coding sequence of modified Ls20ox1-B (SEQ ID NO.: 12). The substitution of the present invention is marked in bold and underlined.

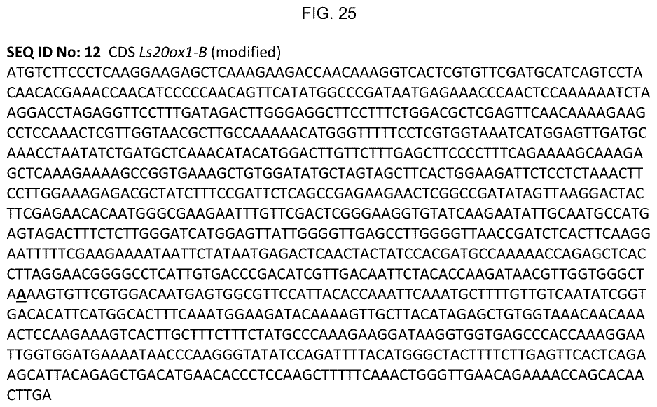

### DETAILED DESCRIPTION OF THE INVENTION

The present invention provides a shade tolerant lettuce plant, wherein either the LsKO2 gene, or the Ls20ox1-B gene, or both genes, is modified such that the protein product of either one or both of these genes has a reduced level, a reduced activity or complete absence as compared to a plant wherein said gene or genes are not modified, provided that the modified gene or genes are present homozygously in the plant.

The invention thus relates to a lettuce plant comprising a modified LsKO2 gene and/or a modified Ls20ox1-B gene, the wild-type LsKO2 gene comprising a coding sequence having at least 70% sequence identity to SEQ ID NO.: 9 and the wild-type Ls20ox1-B gene comprising a coding sequence having at least 70% sequence identity to SEQ ID NO.: 11, wherein the modification comprises replacement and/or deletion and/or insertion of nucleotides resulting in an absence of functional KO2 and/or GA20ox1-B protein or wherein the modification results in the absence of the wild-type LsKO2 and/or Ls20ox1-B gene, and wherein the homozygous presence of one or both of the modified LsKO2 and Ls20ox1-B genes in the plant or the homozygous absence of one or both of the wild-type LsKO2 and/or Ls20ox1-B genes from the plant confers shade tolerance to the plant as compared to a plant comprising the wild-type LsKO2 and Ls20ox1-B gene and not showing shade tolerance.

The modified LsKO2 gene can be as comprised in the genome of Lactuca sativa plant, representative seed of which was deposited under accession number NCIMB 43547. The plant can comprise the modified LsKO2 gene of the invention heterozygously, in which case the seeds produced by the plant do not show the reduced shade avoidance syndrome trait but the plant is useful for transferring the modified LsKO2 gene of the invention to another plant. The plant can also comprise the modified LsKO2 gene of the invention homozygously, in which case said plant shows reduced shade avoidance syndrome.

The LsKO2 gene is understood as the gene identified as EVM6.28510, located on chromosome 6 between positions 198192198 and 203437218 of the organism Lactuca sativa, according to the genome assembly Lsat_Salinas_v9 genome assembly, submitted by the Lettuce Genome Resource in 2018 [based on Reyes Chin Wo et al. (2017) Nature Communications 8:14953].

The modified Ls20ox1-B gene can be as comprised in the genome of Lactuca sativa plant representative seed of which was deposited under accession number NCIMB 43548. The plant can comprise the modified Ls20ox1-B gene of the invention heterozygously, in which case the seeds produced by the plant do not show the reduced shade avoidance syndrome trait but the plant is useful for transferring the modified Ls20ox1-B gene of the invention to another plant. The plant can also comprise the modified Ls20ox1-B gene of the invention homozygously, in which case said plant shows reduced shade avoidance syndrome.

The Ls20ox1-B gene is understood as the gene identified as EVM9.9794, located on chromosome 9 between positions 69609879 and 76853453 of the organism Lactuca sativa, according to the genome assembly Lsat_Salinas_v9 genome assembly, submitted by the Lettuce Genome Resource in 2018 [based on Reyes Chin Wo et al. (2017) Nature Communications 8:14953].

The term “shade avoidance syndrome” is understood as a set of correlated responses that plants display when they are subjected to the shade of another plant. The shade avoidance syndrome in lettuce is understood as a set of correlated responses that lettuce plants display when they are subjected to the shade of another plant. The shade avoidance syndrome of the vegetative part is understood as a set of responses related to the characteristics which belong to the group of characteristics comprising the length of the hypocotyl, the elongation of the stem, the elongation of the leaf stem, the elongation of the leaf lamina, and the length of the root. The shade avoidance syndrome of the vegetative part in lettuce is understood as a set of responses related to the characteristics which belong to the group of characteristics comprising the elongation of the hypocotyl, the elongation of the stem, the elongation of the leaf stem, the elongation of the leaf lamina, and the length of the root.

The term “shade tolerance” is understood as a statistically significant reduction in the shade avoidance syndrome or a statistically significant reduction in any one of the components of the shade avoidance syndrome in a plant. Ultimately, the shade avoidance syndrome can be completely missing.

In the present application, “shade tolerance” is synonymously referred to as “reduced shade avoidance syndrome”.

The term “reduced” is always measured in relation to the shade avoidance syndrome observed in a control plant or part thereof that has no modification to its LsKO2 and/or Ls20ox1-B gene homologs and is therefore a wild-type plant comprising wild-type LsKO2 and/or Ls20ox1-B gene homologs and does not show reduced shade avoidance syndrome. As used herein, a plant showing a “reduced shade avoidance syndrome” or a plant showing a “reduction of the shade avoidance syndrome” is a plant having a reduced shade avoidance syndrome as compared to the shade avoidance syndrome of a wild-type plant. Therefore, an improvement of the shade avoidance syndrome is defined by a reduced intensity of the shade avoidance syndrome as compared to a plant not comprising a modified LsKO2 and/or Ls20ox1-B gene homolog. The reduction of the shade avoidance syndrome can be assessed qualitatively and quantitatively.

A reduced shade avoidance syndrome is quantitatively determined in a young plant assay. In this assay, seeds of each genotype to be tested are germinated in trays and grown under so-called greenhouse conditions (with the average conditions being about 16 h day time at about 20° C./about 8h night time at about 17° C.) until the cotyledons are fully expanded and the first true leaves become visible. Then trays are covered with green filter which uniformly reduces the R/FR light reaching the plants to a value below 0.30. The green filter is suitably a Lee Filter Roll 122 Fern Green plastic foil sheet. After 26 days of growth under low R/FR light conditions, the hypocotyl length of the young plants is measured. As a comparison, the hypocotyl length of a plant grown under normal R/FR conditions (R/FR>1) is between 1 and 5 mm.

As used herein, a shade tolerant plant is a plant that, when as young plant exposed to the above described assay, has a hypocotyl length of less than 71% of the hypocotyl length of a shade non-tolerant plant.

A plant of the invention will in the above described assay typically show a hypocotyl length between 5 and 15 mm, after 26 days of growth, while a shade non-tolerant plant will typically show a hypocotyl length above 22 mm, after 26 days of growth.

As used herein, the term “lettuce plant of the invention” or “plant of the invention” is intended to refer to a lettuce (Lactuca sativa) plant comprising the modified LsKO2 gene of the invention or a lettuce plant comprising the modified Ls20ox1-B gene of the invention, or a lettuce plant comprising both the modified LsKO2 gene of the invention and the modified Ls20ox1-B gene of the invention. Preferably, in the plant of the invention said modification is as comprised in the genome of a Lactuca sativa plant representative seed of which was deposited under accession number NCIMB 43547 and NCIMB 43548.

A “lettuce plant of the invention” or a “plant of the invention” can be a lettuce plant of any type, and is preferably an agronomically elite lettuce plant.

In the context of this invention an “agronomically elite lettuce” plant is a plant having a genotype that results in an accumulation of distinguishable and desirable agronomic traits which allow a producer to harvest a product of commercial significance.

As used herein, a “plant of an inbred line” is a plant of a population of plants that is the result of three or more rounds of selfing, or backcrossing, or which plant is a doubled haploid. An inbred line can e.g. be a parent line used for the production of a commercial hybrid.

In one embodiment, the shade tolerance is the result of a modification, in other word, alteration, of the endogenous LsKO2 gene, leading to a reduction or absence of endogenous expression of the KO2 protein in the lettuce plant.

The modified LsKO2 gene of the plant of the invention comprises one or more nucleotides replaced, inserted and/or deleted relative to the wild-type, whereby said one or more replaced, inserted and/or deleted nucleotides result in an absence of functional KO2 protein. The absence of functional KO2 protein can be due to the absence of LsKO2 RNA or a significantly decreased LsKO2 RNA level, resulting in a complete absence or a reduced and biologically inadequate level of KO2 protein. The absence of functional KO2 protein can also mean an absence of one or more of the functional domains of the KO2 protein, resulting in a modified KO2 protein that cannot perform its function as an oxidase enzyme. The absence of functional KO2 protein can further mean that the modified protein has gained certain amino acids, destroying the wild-type functionality of the protein. More specifically, the absence of functional protein can further mean that the protein has lost a protein-protein and/or protein-DNA interaction site.

In particular, the present invention provides a plant comprising a modified LsKO2 gene, wherein the modified LsKO2 gene comprises a mutation in SEQ ID NO.: 9, or in a homologous sequence having in order of increased preference at least 70%, 75%, 80%, 85%, 90%, 91% 92%, 93%, 94%, 95%, 96%, 97%, 98%, 99% sequence identity to SEQ ID NO.: 9.

The modified LsKO2 gene of the plant of the invention can comprise, in particular, a mutation on or before position 1102 of SEQ ID NO.: 9, or on a corresponding position of a homologous sequence having in order of increased preference at least 70%, 75%, 80%, 85%, 90%, 91% 92%, 93%, 94%, 95%, 96%, 97%, 98%, 99% sequence identity to SEQ ID NO.: 9.

As used herein, sequence identity is the percentage of nucleotides or amino acids that is identical between two sequences after proper alignment of those sequences. The person skilled in the art is aware of how to align sequences, for example by using a sequence alignment tool such as BLAST®, which can be used for both nucleotide sequences and protein sequences. To obtain the most significant result, the best possible alignment that gives the highest sequence identity score should be obtained. The percentage sequence identity is calculated through comparison over the length of the shortest sequence in the assessment, whereby in the present case a sequence represents a gene that at least comprises a start codon and a stop codon, or a complete protein encoded by such a gene.

The invention further relates to a plant comprising a modified LsKO2 gene comprising a mutation leading to amino acid change, wherein amino acid change results in an absence of the wild-type KO2 protein.

In particular, the invention relates to a plant comprising a modified LsKO2 gene wherein the LsKO2 gene comprises a mutation leading to a premature stop codon, and wherein the premature stop codon results in an absence of functional KO2 protein. Preferably, the premature stop codon is located within or before the part encoding the cytochrome p450 domain of the KO2 protein.

In particular, the present invention provides a plant comprising a modified LsKO2 gene, wherein the modified gene comprises a mutation on or before position 1102 of SEQ ID NO.: 9, or wherein the modified KO2 protein is truncated, on or before position 368 of SEQ ID NO.: 3, or on a corresponding position of a homologous amino acid sequence having in order of increased preference at least 70%, 75%, 80%, 85%, 90%, 91% 92%, 93%, 94%, 95%, 96%, 97%, 98%, 99% sequence identity to SEQ ID NO.: 3.

In one embodiment, the protein translated from the modified LsKO2 gene of the plant of the invention comprises a premature stop codon that renders the encoded protein partly or entirely nonfunctional. In a preferred embodiment, the one or more nucleotides that are replaced, inserted and/or deleted in the modified LsKO2 gene of the plant of the invention relative to the wild-type are at position 1102 of SEQ ID NO.: 9, resulting in a premature stop codon that leads to an absence of a functional protein. In a preferred embodiment, the modified LsKO2 gene of the plant of the invention comprises a substitution of a cytosine by a thymine at position 1102 of SEQ ID NO.: 9, leading to the termination of the protein translation process at amino acid position 368 (Q368*) of the encoded KO2 protein.

In yet a preferred embodiment, the plant of the invention comprises a modified LsKO2 gene characterized by a coding sequence of SEQ ID NO.: 10, or a sequence encoding a protein having SEQ ID NO.: 4.

In the genome of a lettuce plant representative seed of which was deposited under accession number NCIMB 43547 there is a substitution of a cytosine by a thymine at the genomic position 200841726 on lg6. This one base pair substitution leads to the early termination of the synthesis of the encoded protein sequence (SEQ ID NO.: 4). Whereas the size of the wild-type KO2 protein is 512 amino acids (SEQ ID NO.: 3), the modified KO2 protein (SEQ ID NO.:4) is 144 amino acids shorter, being only 368 amino acids long. The truncated KO2 protein lacks a substantial part of its p450 functional domain. The mutant protein is thus not functional.

The modified KO2 gene of the plant of the invention confers shade tolerance to the plant when present homozygously.

In another embodiment of the invention, the shade tolerance is the result of a modification of the Ls20ox1-B gene, leading to a lettuce plant in which the endogenous expression of the GA20ox-1B protein is reduced or absent.

The modified Ls20ox1-B gene of the plant of the invention comprises one or more nucleotides replaced, inserted and/or deleted relative to the wild-type, and said one or more replaced, inserted and/or deleted nucleotides result in an absence of functional GA20ox-1B protein. The absence of functional GA20ox-1B protein can be due to the absence of Ls20ox1-B RNA or a significantly decreased Ls20ox1-B RNA level, resulting in a complete absence or a reduced and biologically inadequate level of GA20ox-1B protein. The absence of functional GA20ox-1B protein can also mean an absence of one or more of the functional domains of the GA20ox-1B protein, resulting in a modified GA20ox-1B protein that cannot perform its function as an oxidase enzyme. The absence of functional GA20ox-1B protein can further mean that the modified protein has gained certain amino acids, destroying the wild-type functionality of the protein. More specifically, the absence of functional protein can further mean that the protein has lost a protein-protein and/or protein-DNA interaction site.

In particular, the present invention provides a plant comprising a modified Ls20ox1-B gene wherein the modified gene comprises a mutation in SEQ ID NO.: 11, or in a homologous sequence having in order of increased preference at least 70%, 75%, 80%, 85%, 90%, 91% 92%, 93%, 94%, 95%, 96%, 97%, 98%, 99% sequence identity to SEQ ID NO.: 11.

The modified Ls20ox1-B gene of the plant of the invention can comprise, in particular, a nucleotide substitution of a guanine by an adenine at position 766 of SEQ ID NO.: 11, or on a corresponding position of a homologous sequence having in order of increased preference at least 70%, 75%, 80%, 85%, 90%, 91% 92%, 93%, 94%, 95%, 96%, 97%, 98%, 99% sequence identity to SEQ ID NO.: 11.

The invention further relates to a plant comprising a modified Ls20ox1-B gene comprising a mutation leading to a non-conservative amino acid substitution, wherein the non-conservative amino acid substitution results in an absence of the wild-type GA20ox-1B protein.

In particular, the present invention also provides a plant comprising a modified Ls20ox1-B gene, wherein the modified gene encodes a protein having a non-conservative amino acid substitution in position 268 of SEQ ID NO.: 7 or on a corresponding position of a homologous amino acid sequence having in order of increased preference at least 70%, 75%, 80%, 85%, 90%, 91% 92%, 93%, 94%, 95%, 96%, 97%, 98%, 99% sequence identity to SEQ ID NO.: 7.

The invention further provides a plant comprising a modified Ls20ox1-B gene wherein the Ls20ox1-B gene comprises a non-conservative amino acid substitution as a result of a substitution of a guanine by an adenine at position 766 of SEQ ID NO.: 11, or on a corresponding position of a homologous sequence having in order of increased preference at least 70%, 75%, 80%, 85%, 90%, 91% 92%, 93%, 94%, 95%, 96%, 97%, 98%, 99% sequence identity to SEQ ID NO.: 11, or wherein the modified gene encodes a protein having a non-conservative amino acid substitution at position 268 of SEQ ID NO.: 7, or on a corresponding position of a homologous amino acid sequence having in order of increased preference at least 70%, 75%, 80%, 85%, 90%, 91% 92%, 93%, 94%, 95%, 96%, 97%, 98%, 99% sequence identity to SEQ ID NO.: 7.

Preferably, the amino acid substitution is located within or before the part encoding one of the functional domains of the GA20ox-1B protein.

Therefore, the present invention also provides a plant comprising a modified Ls20ox1-B gene, wherein the modified gene comprises a mutation on or before position 766 of SEQ ID NO.: 11, or wherein the modified GA20ox-1B protein is truncated, on or before position 268 of SEQ ID NO.: 7.

In a preferred embodiment, the modified Ls20ox1-B gene of the plant of the invention comprises a coding sequence having SEQ ID NO.: 12, or a sequence encoding a protein having SEQ ID NO.: 8.

The modified Ls20ox1-B gene of the plant of the invention, when homozygously present in a plant, in particular a plant of the Asteraceae (Compositae) plant family and more in particular a lettuce plant (Lactuca sativa), leads to a decreased shade avoidance syndrome, in other words, it confers shade tolerance to the plant.

In one embodiment, the protein translated from the modified Ls20ox1-B gene of the plant of the invention comprises a non-conservative amino acid substitution that renders the protein partly or entirely nonfunctional. In a preferred embodiment, the one or more nucleotides that are replaced, inserted and/or deleted in the modified Ls20ox1-B gene of the invention relative to the wild-type are at position 766 of SEQ ID NO.: 11, resulting in a non-conservative amino acid substitution that leads to an absence of the wild-type GA20ox-1B protein. In a preferred embodiment, the modified Ls20ox1-B gene of the invention comprises a substitution of a guanine by an adenine at position 766 of SEQ ID NO.: 11, resulting in a premature stop codon at amino acid position 268 (E268K) in the sequence of the translated GA20ox-1B protein.

In the genome of a lettuce plant representative seed of which was deposited under accession number NCIMB 43548 there is a substitution of a guanine by an adenine at the genomic position 72575669 on lg9. This one base pair substitution leads to a non-conservative amino acid change at position 268 of the encoded GA20ox-1B protein (the modified protein is identified as SEQ ID NO.: 8). The replacement of a negatively charged amino acid glutamic acid by a positively charged amino acid lysine within the Fe2OG-dioxygenase functional domain leads to a disturbance in the folding and function of the resulting GA20ox-1B protein. The functionality of the mutant GA20ox-1B protein is thus severely affected.

The modified Ls20ox1-B gene of the plant of this invention confers shade tolerance to the plant when present homozygously.

Thus, in one embodiment, the plant of the invention comprises the modified LsKO2 gene and/or modified Ls20ox1-B gene of the invention, preferably in homozygous state, wherein the modification in the LsKO2 gene and/or Ls20ox1-B gene is non-naturally occurring.

In a further embodiment, the plant of the invention comprises the modified LsKO2 gene and/or modified Ls20ox1-B gene of the invention, preferably in homozygous state, wherein the modification of the LsKO2 gene and/or Ls20ox1-B gene is the result of humanly induced mutagenesis, wherein the induced mutagenesis can be a form of random mutagenesis, or a form of site-directed mutagenesis.

The invention also encompasses a lettuce seed, comprising the modified LsKO2 gene of the invention, wherein the plant grown from said seed shows reduced shade avoidance syndrome as a result of the homozygous presence of the modified LsKO2 gene.

The invention also encompasses a lettuce seed, comprising the modified Ls20ox1-B gene of the invention, wherein the plant grown from said seed shows reduced shade avoidance syndrome as a result of the homozygous presence of the modified Ls20ox1-B gene.

The invention also encompasses a lettuce seed, comprising the modified LsKO2 gene and the modified Ls20ox1-B gene of the invention, wherein the plant grown from said seed shows reduced shade avoidance syndrome as a result of the homozygous presence of the modified LsKO2 gene and the modified Ls20ox1-B gene.

The invention further relates to any part of the lettuce plant of the invention, wherein the plant part comprises the modified LsKO2 gene of the invention and optionally further comprises the modified Ls20ox1-B gene of the invention.

The invention further relates to any part of the lettuce plant of the invention, wherein the plant part comprises the modified Ls20ox1-B gene of the invention.

Moreover, the invention also relates to a food product or a processed food product comprising the plant of the invention or any part thereof. The food product can have undergone one or more processing steps. Such a processing step might comprise but is not limited to any one of the following treatments or combinations thereof: cutting, washing, juicing, cooking, cooling or preparing a salad mixture comprising the plant of the invention or any part thereof. The processed form that is obtained is also part of this invention.

The invention further relates to a cell of a plant of the invention. Such a cell can either be in isolated form or a part of the complete plant or parts thereof and still constitutes a cell of the invention because such a cell harbors the genetic information that imparts the reduced shade avoidance syndrome to a plant of the invention. Each cell of a plant of the invention carries the genetic information that leads to the reduced shade avoidance syndrome of the invention. A cell of the invention can also be a regenerable cell that can regenerate into a new plant of the invention.

The presence of genetic information as used herein is the presence of the modified LsKO2 gene of the invention and/or the presence of the modified Ls20ox1-B gene of the invention.

The invention further relates to plant tissue of a plant of the invention, which comprises the modified LsKO2 gene of the invention and/or the modified Ls20ox1-B gene of the invention. The tissue can be undifferentiated tissue or already differentiated tissue. Undifferentiated tissue is for example a stem tip, an anther, a petal, or pollen, and can be used in micropropagation to obtain new plantlets that are grown into new plants of the invention. The tissue can also be grown from a cell of the invention.

The invention moreover relates to progeny of a plant, a cell, a tissue, or a seed of the invention, which progeny comprises the modified LsKO2 gene of the invention and/or the modified Ls20ox1-B gene of the invention. Such progeny can in itself be a plant, a cell, a tissue, or a seed. The progeny can in particular be progeny of a plant of the invention deposited under NCIMB accession numbers 43547 or 43548. As used herein “progeny” is intended to mean the first and all further descendants from a cross with a plant of the invention, wherein a cross comprises a cross with itself or a cross with another plant, and wherein a descendant that is determined to be progeny comprises the modified LsKO2 gene of the invention and/or the modified Ls20ox1-B gene of the invention. Progeny also encompasses material that is obtained by vegetative propagation or another form of multiplication. Preferably, the progeny plant shows reduced shade avoidance syndrome as a result of the homozygous presence of the modified LsKO2 gene of the invention and/or the modified Ls20ox1-B gene of the invention.

The invention also relates to propagation material capable of developing into and/or being derived from a plant of the invention, wherein the propagation material comprises the modified LsKO2 gene of the invention and/or the modified Ls20ox1-B gene of the invention, and wherein the propagation material is selected from a group consisting of a microspore, a pollen, an ovary, an ovule, an embryo, an embryo sac, an egg cell, a cutting, a root, a root tip, a hypocotyl, a cotyledon, a stem, a leave, a flower, an anther, a seed, a meristematic cell, a protoplast and a cell, or a tissue culture thereof.

In another aspect, the present invention relates to the modified LsKO2 gene which imparts shade tolerance to the plant carrying said gene, wherein the LsKO2 gene is modified as compared to the wild-type, which is identified as SEQ ID NO.: 1, has a coding sequence according to SEQ ID NO.: 9, and encodes a protein having a sequence according to SEQ ID NO.: 3, or the wild-type of which encodes a protein having in order of increased preference at least 70%, 75%, 80%, 85%, 90%, 91% 92%, 93%, 94%, 95%, 96%, 97%, 98%, 99% sequence identity to SEQ ID NO.: 3, and wherein the modified LsKO2 gene comprises one or more nucleotides replaced, inserted and/or deleted relative to the wild-type, and wherein said one or more replaced, inserted and/or deleted nucleotides result in an absence of functional KO2 protein.

In a particular embodiment, the one or more nucleotides that are replaced, inserted and/or deleted in the modified LsKO2 gene relative to the wild-type result in a mutation leading to a premature stop codon in the translated protein. Preferably, the premature stop codon is located within or before the part encoding the cytochrome p450 domain of the KO2 protein.

In another particular embodiment, the modified LsKO2 gene comprises a mutation at position 1102 of SEQ ID NO.: 9, resulting in a premature stop codon that leads to an absence of a functional protein.

In a further particular embodiment, the modified LsKO2 gene comprises a substitution of a cytosine by a thymine at position 1102 of SEQ ID NO.: 9, resulting in a premature stop codon, which in turn leads to the early termination of protein synthesis at amino acid position 368 (Q368*) in the translated KO2 protein.

In a further embodiment, the modified LsKO2 gene comprises a mutation before position 1102 of SEQ ID NO.: 9, resulting in a premature stop codon that leads to an absence of a functional protein.

In a further particular embodiment, the protein translated from the modified LsKO2 gene is partly or entirely nonfunctional.

In another particular embodiment, the modified LsKO2 gene is as comprised in the genome of seeds of which a representative sample is deposited under accession number NCIMB 43547.

In yet another aspect, the present invention relates to the modified Ls20ox1-B gene which imparts shade tolerance to the plant carrying said gene, wherein the Ls20ox1-B gene is modified as compared to the wild-type according to SEQ ID NO.: 5, has a coding sequence according to SEQ ID NO.: 11, and encodes the GA20ox1-B protein according to SEQ ID NO.: 7, or the wild-type of which encodes a protein having in order of increased preference at least 70%, 75%, 80%, 85%, 90%, 91% 92%, 93%, 94%, 95%, 96%, 97%, 98%, 99% sequence identity to SEQ ID NO.: 7, and wherein the modified Ls20ox1-B gene comprises one or more nucleotides replaced, inserted and/or deleted relative to the wild-type, and wherein said one or more replaced, inserted and/or deleted nucleotides result in an absence of functional GA20ox-1B protein.

In a particular embodiment, the one or more nucleotides that are replaced, inserted and/or deleted in the modified Ls20ox1-B gene relative to the wild-type result in a mutation leading to a non-conservative amino acid substitution, wherein the non-conservative amino acid substitution results in an absence of the wild-type GA20ox-1B protein. Preferably, the amino acid substitution is located within or before the part encoding one of the Fe2OG-dioxygenase functional domain of the GA20ox-1B protein.

In another particular embodiment, the modified Ls20ox1-B gene comprises a mutation on or before position 766 of SEQ ID NO.: 11, or wherein the modified GA20ox-1B protein is truncated, on or before position 268 of SEQ ID NO.: 7.

In one embodiment, the modified Ls20ox1-B gene of the invention comprises a coding sequence having SEQ ID NO.: 12, or a sequence encoding a protein having SEQ ID NO.: 8.

In another particular embodiment, the modified Ls20ox1-B gene is as comprised in the genome of seeds of which a representative sample is deposited under accession number NCIMB 43548.

The invention further relates to use of the modified LsKO2 gene and the modified Ls20ox1-B gene of the invention for producing a plant that shows reduced shade avoidance syndrome. The plant that shows reduced shade avoidance syndrome can be produced by introduction of the modified LsKO2 gene and the modified Ls20ox1-B gene into its genome, in particular by means of mutagenesis or introgression, or combinations thereof.

The invention also relates to use of the plant of the invention for the production of plants with reduced shade avoidance syndrome.

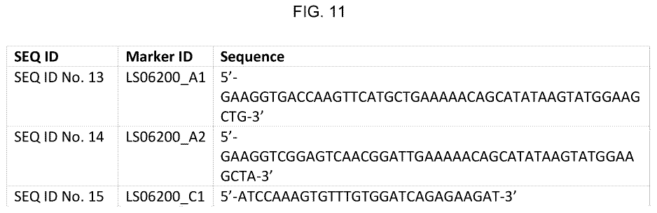

The invention further relates to a marker for the identification of the modified LsKO2 gene, wherein the marker sequence detects a SNP in SEQ ID NO.: 2. An example of such a marker is marker LS06200_C1 (SEQ ID NO.: 15, FIG. 11).

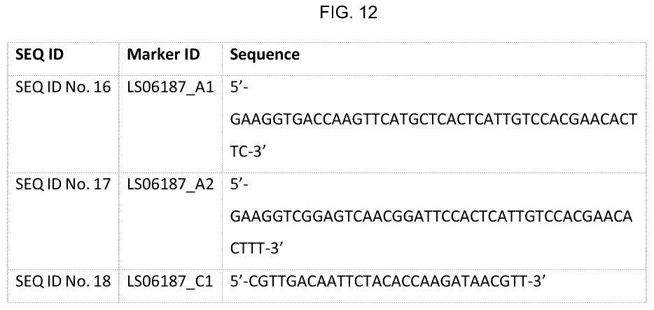

The invention further relates to a marker for the identification of the modified Ls20ox1-B gene, wherein the marker sequence detects a SNP in SEQ ID NO.: 6. An example of such a marker is marker LS06187_C1 (SEQ ID NO.: 18, FIG. 12).

Use of these markers alone or in combination for identification and/or selection of a lettuce plant with reduced shade avoidance syndrome is also part of this invention. The invention further relates to a method for selecting a lettuce plant that shows reduced shade avoidance syndrome, comprising identifying the presence of a modification in the LsKO2 gene and/or a modification in the Ls20ox1-B gene, optionally checking the shade avoidance syndrome, and selecting a plant that homozygously comprises said modification as a plant that shows reduced shade avoidance syndrome. The identification of the presence of a modification in the LsKO2 gene can be performed by using the marker as defined above.

The identification of the presence of a modification in the Ls20ox1-B gene can be performed by using the marker as defined above.

The invention further relates to a method for producing a shade tolerant lettuce plant, comprising the modification of the wild-type of the LsKO2 gene and/or Ls20ox1-B gene, wherein the modification results in an increased shade tolerance in the plant. Said method comprises the introduction of a deletion, a substitution, or an insertion in the coding sequence of an LsKO2 gene and/or Ls20ox1-B gene.

In a particular embodiment, the plant of the invention is not exclusively obtained by means of an essentially biological process.

Introduction of the modified LsKO2 gene and/or modified Ls20ox1-B gene of the invention can also be done through introgression from a plant comprising said modified LsKO2 gene and/or modified Ls20ox1-B gene, for example from a plant that was deposited as NCIMB 43547 and/or NCIMB 43548, or from progeny thereof, or from another plant that is shade tolerant, and in which the modified LsKO2 gene and/or modified Ls20ox1-B gene of the invention was identified. Breeding methods such as crossing and selection, backcrossing, recombinant selection, or other breeding methods that result in the transfer of a genetic sequence from a resistant plant to a susceptible plant can be used. A resistant plant can be of the same species or of a different and/or wild species. Difficulties in crossing between species can be overcome through techniques known in the art such as embryo rescue, or cis-genesis can be applied. Progeny of a deposit can be sexual or vegetative descendants of that deposit, which can be selfed and/or crossed, and can be of an F1, F2, or further generation as long as the descendants of the deposit still comprise the modified gene the invention as present in seed of that deposit. A plant produced by such method is also a part of the invention.

The present invention also relates to a method for the production of a shade tolerant lettuce plant, said method comprising:

a) crossing a plant comprising the modified LsKO2 gene of the invention with a plant not comprising said modified LsKO2 gene, or crossing a plant comprising the modified Ls20ox1-B gene of the invention with a plant not comprising said modified Ls20ox1-B gene, or crossing a plant comprising both the modified LsKO2 gene of the invention and the modified Ls20ox1-B gene of the invention with a plant not comprising said modified LsKO2 and Ls20ox1-B genes; b) optionally performing one or more rounds of selfing and/or crossing a plant resulting from step a) to obtain a further generation population; c) selecting from the population a plant that comprises either the modified LsKO2 gene or the modified Ls20ox1-B gene or both of said modified genes, that alone or together confer shade tolerance to the plant.

The present invention relates to a method for the production of a shade tolerant lettuce plant, said method comprising: a) crossing a plant comprising the modified LsKO2 gene of the invention with a plant not comprising said modified LsKO2 gene;

b) backcrossing the plant resulting from step a) with the parent not comprising the modified LsKO2 gene for at least three generations;

c) selecting from the third or higher backcross population a plant that comprises the modified LsKO2 gene that confers reduced shade avoidance syndrome to the plant.

The present invention relates to a method for the production of a shade tolerant lettuce plant, said method comprising:

a) crossing a plant comprising the modified Ls20ox1-B gene of the invention with a plant not comprising said modified Ls20ox1-B gene;

b) backcrossing the plant resulting from step a) with the parent not comprising the modified Ls20ox1-B gene for at least three generations;

c) selecting from the third or higher backcross population a plant that comprises the modified Ls20ox1-B gene that confers reduced shade avoidance syndrome to the plant.

The presence of a modified LsKO2 gene and/or modified KO2 protein, optionally in isolated form, leading to a reduced shade avoidance syndrome can be detected using routine methods known to the skilled person such as RT-PCR, PCR, antibody-based assays, sequencing and genotyping assays, or combinations thereof. Such methods can be used to determine for example, a reduction of the expression of the wild-type LsKO2 gene, a reduction of the expression of wild-type KO2 protein, the presence of a modified mRNA, cDNA or genomic DNA encoding a modified KO2 protein, or the presence of a modified KO2 protein, in plant material or plant parts, or DNA or RNA or protein derived therefrom.

The presence of a modified Ls20ox1-B gene and/or modified GA20ox1-B protein, optionally in isolated form, leading to a reduced shade avoidance syndrome can be detected using routine methods known to the skilled person such as RT-PCR, PCR, antibody-based assays, sequencing and genotyping assays, or combinations thereof. Such methods can be used to determine for example, a reduction of the expression of the wild-type Ls20ox1-B gene, a reduction of the expression of wild-type GA20ox1-B protein, the presence of a modified mRNA, cDNA or genomic DNA encoding a modified GA20ox1-B protein, or the presence of a modified GA20ox1-B protein, in plant material or plant parts, or DNA or RNA or protein derived therefrom.

Modifications or mutations of the wild-type LsKO2 gene and/or modifications or mutations of the wild-type Ls20ox1-B gene can be introduced randomly by means of one or more chemical compounds, such as EMS, nitrosomethylurea, hydroxylamine, proflavine, N-methly-N-nitrosoguanidine, N-ethyl-N-nitrosourea, N-methyl-Nnitro-nitrosoguanidine, diethyl sulphate, ethylene imine, sodium azide, formaline, urethane, phenol and ethylene oxide, and/or by physical means, such as UV-irradiation, fast neutron exposure, X rays, gamma irradiation, and/or by insertion of genetic elements, such as transposons, T-DNA, retroviral elements.

Modifying the wild-type LsKO2 gene and/or modifying the wild-type Ls20ox1-B gene could also comprise the step of targeted genome editing, wherein the sequence of the wild-type LsKO2 gene or the sequence of the wild-type Ls20ox1-B gene is modified, or wherein the wild-type LsKO2 gene or the wild-type Ls20ox1-B gene is replaced by, respectively, another LsKO2 or Ls20ox1-B gene that is modified. This can be achieved by means of any method known in the art for modifying DNA in the genome of a plant, or by means of methods for gene replacement. Such methods include genome editing techniques and homologous recombination.

Homologous recombination allows the targeted insertion of a nucleic acid construct into a genome, and the targeting is based on the presence of unique sequences that flank the targeted integration site. For example, the wild-type locus of a LsKO2 gene could be replaced by a nucleic acid construct comprising a modified LsKO2 gene or the wild-type locus of a Ls20ox1-B gene could be replaced by a nucleic acid construct comprising a modified Ls20ox1-B gene.

Modifying a wild-type LsKO2 or Ls20ox1-B gene can involve inducing double strand breaks in DNA using zinc-finger nucleases (ZFN), TAL (transcription activator-like) effector nucleases (TALEN), Clustered Regularly Interspaced Short Palindromic Repeats/CRISPR-associated nuclease (CRISPR/Cas nuclease), or homing endonucleases that have been engineered to make double-strand breaks at specific recognition sequences in the genome of a plant, another organism, or a host cell.

TAL effector nucleases (TALENs) can be used to make double-strand breaks at specific recognition sequences in the genome of a plant for gene modification or gene replacement through homologous recombination. TAL effector nucleases are a class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, for example, Fok I. The unique, modular TAL effector DNA binding domain allows for the design of proteins with potentially any given DNA recognition specificity.

Thus, the DNA binding domains of the TAL effector nucleases can be engineered to recognize specific DNA target sites and thus, used to make double-strand breaks at desired target sequences.

ZFNs can be used to make double-strand breaks at specific recognition sequences in the genome of a plant for gene modification or gene replacement through homologous recombination. The Zinc Finger Nuclease (ZFN) is a fusion protein comprising the part of the Fok I restriction endonuclease protein responsible for DNA cleavage and a zinc finger protein which recognizes specific, designed genomic sequences and cleaves the double-strand DNA at those sequences, thereby producing free DNA ends (Urnov et al, 2010, Nat. Rev. Genet. 11: 636-46; Carroll, 2011, Genetics 188: 773-82).

The CRISPR/Cas nuclease system can also be used to make double-strand breaks at specific recognition sequences in the genome of a plant for gene modification or gene replacement through homologous recombination. The CRISPR/Cas nuclease system is an RNA-guided DNA endonuclease system performing sequence-specific double-strand breaks in a DNA segment homologous to the designed RNA. It is possible to design the specificity of the sequence (Jinek et al, 2012, Science 337: 816-821; Cho et al, 2013, Nat. Biotechnol. 31: 230-232; Cong et al, 2013, Science 339: 819-823; Mali et al., 2013, Science 339: 823-826; Feng et al, 2013, Cell Res. 23: 1229-1232). Cas9 is an RNA-guided endonuclease that has the capacity to create double-strand breaks in DNA in vitro and in vivo, also in eukaryotic cells. It is part of an RNA-mediated adaptive defence system known as Clustered Regularly Interspaced Short Palindromic Repeats (CRISPR) in bacteria and archaea. Cas9 gets sequence-specificity when it associates with a guide RNA molecule, which can target sequences present in an organism's DNA based on their sequence. Cas9 requires the presence of a Protospacer Adjacent Motif (PAM) immediately following the DNA sequence that is targeted by the guide RNA. The Cas9 enzyme has been first isolated from Streptococcus pyogenes (SpCas9), but functional homologues from many other bacterial species have been reported, such as Neisseria meningitides, Treponema denticola, Streptococcus thermophilus, Francisella novicida, Staphylococcus aureus, etcetera. For SpCas9, the PAM sequence is 5′-NGG-3′, whereas various Cas9 proteins from other bacteria have been shown to recognize different PAM sequences. In nature, the guide RNA is a duplex between crRNA and tracrRNA, but a single guide RNA (sgRNA) molecule comprising both crRNA and tracrRNA has been shown to work equally well (Jinek et al, 2012, Science 337: 816-821). The advantage of using an sgRNA is that it reduces the complexity of the CRISPR-Cas9 system down to two components, instead of three. For use in an experimental setup (in vitro or in vivo) this is an important simplification.

An alternative to the Cas9 nuclease is, for example, the use of Cas9 nickases. Nickases create single-strand rather than a double-strand breaks. Nickases can also be used as a double nickase system.

A further alternative to the Cas9 nuclease is, for example, Cpf1 (also known as Cas12a), which does not need a tracrRNA to function and creates sticky end cuts in the DNA, contrarily to Cas9 which creates blunt ends.

On the one hand, genetic modification techniques can be applied to express a site-specific nuclease, such as an RNA-guided endonuclease and/or guide RNAs, in eukaryotic cells. One or more DNA constructs encoding an RNA-guided endonuclease and at least one guide RNA can be introduced into a cell or organism by means of stable transformation (wherein the DNA construct is integrated into the genome) or by means of transient expression (wherein the DNA construct is not integrated into the genome, but it expresses an RNA-guided endonuclease and at least one guide RNA in a transient manner). This approach requires the use of a transformation vector and a suitable promoter for expression in said cell or organism. There is also an alternative, “DNA-free” delivery method of CRISPR-Cas components into intact plants that does not involve the introduction of DNA constructs into the cell or organism.

For example, introducing the mRNA encoding Cas9 into a cell or organism has been described, after in vitro transcription of said mRNA from a DNA construct encoding an RNA-guided endonuclease, together with at least one guide RNA. This approach does not require the use of a transformation vector and a suitable promoter for expression in said cell or organism.

Another known approach is the in vitro assembly of ribonucleoprotein (RNP) complexes, comprising an RNA-guided endonuclease protein (for example Cas9) and at least one guide RNA, and subsequently introducing the RNP complex into a cell or organism. In animals and animal cell and tissue cultures, RNP complexes have been introduced by means of, for example, injection, electroporation, nanoparticles, vesicles, and with the help of cell-penetrating peptides. In plants, the use of RNPs has been demonstrated in protoplasts, for example with polyethylene glycol (PEG) transfection (Woo et al, 2015, Nat. Biotech. 33: 1162-1164). After said modification of a genomic sequence has taken place, the protoplasts or cells can be used to produce plants that harbor said modification in their genome, using any plant regeneration method known in the art (such as in vitro tissue culture).

Breaking DNA using site specific nucleases, such as, for example, those described herein above, can increase the rate of homologous recombination in the region of the breakage. Thus, coupling of such effectors as described above with nucleases enables the generation of targeted changes in genomes which include additions, deletions and other modifications.

The invention further relates to the use of the plant as claimed for the production of lettuce seeds.

Also part of this invention is the use of the modified LsKO2 gene and/or the use of the modified Ls20ox1-B gene for producing a shade tolerant plant. The shade tolerant plant can either be produced by mutagenesis of the endogenous wildtype LsKO2 gene and/or Ls20ox1-B gene to produce a modified enodogenous LsKO2 gene and/or Ls20ox1-B gene or by introgression of the modified gene(s), or combinations thereof.

The invention is also directed to use of a marker the sequence of which is as provided in FIG. 11 and FIG. 12 for the identification of the modified LsKO2 and/or Ls20ox1-B, respectively.

This invention also provides a method of identifying molecular markers linked to shade tolerance, the sequence of which is as provided in FIG. 11 and FIG. 12, the method comprising:

a) isolating DNA from a plant and from one or both parents of said plant;

b) screening for molecular markers in a region of said DNA at or near the sequence corresponding to SEQ ID No 9 and/or SEQ ID No 11;

c) determining co-inheritance of said markers from one or both parents of said plant.

d) identifying said molecular markers in said region.

The invention is further directed to a method for producing a shade tolerant plant, said method comprising

a) crossing a plant comprising a modified LsKO2 and/or Ls20ox1-B gene with another plant to obtain an F1 population;

b) optionally performing one or more rounds of selfing and/or crossing a plant from the F1 to obtain a further generation population;

c) selecting from the population a plant that comprises the modified LsKO2 and/or Ls20ox1-B gene and is shade tolerant.

The lettuce plant that shows shade tolerance is suitably produced by introduction of the modified gene, in particular by means of mutagenesis or introgression, or combinations thereof.

The invention also provides a method for determining the genotype of a plant as claimed, comprising the steps of obtaining a sample of nucleic acids from said plant, comparing said nucleic acids to a sample of nucleic acids obtained from a reference plant comprising the wild-type of LsKO2 and/or Ls20ox1-B gene homologs, and detecting a polymorphism between the two nucleic acid samples, wherein the detected polymorphism is indicative of the presence of said modified homolog.

Although the present invention and its advantages have been described in detail, it should be understood that various changes, substitutions and alterations can be made herein without departing from the spirit and scope of the invention as defined in the appended claims.

The present invention will be further illustrated in the following Examples, which are given for illustration purposes only and are not intended to limit the invention in any way.

### EXAMPLES

### Example 1

### High Throughput Screening for Shade Tolerance

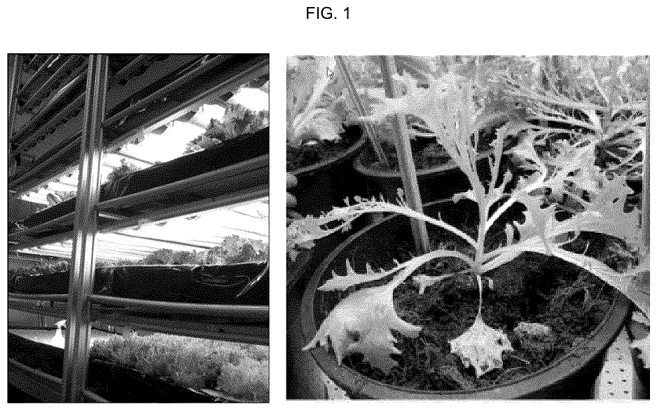

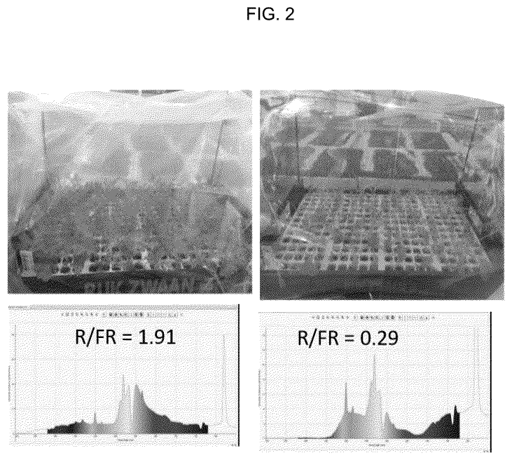

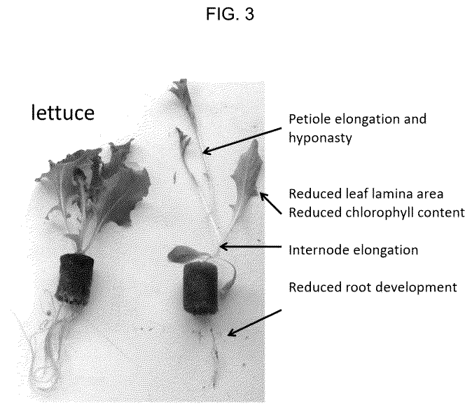

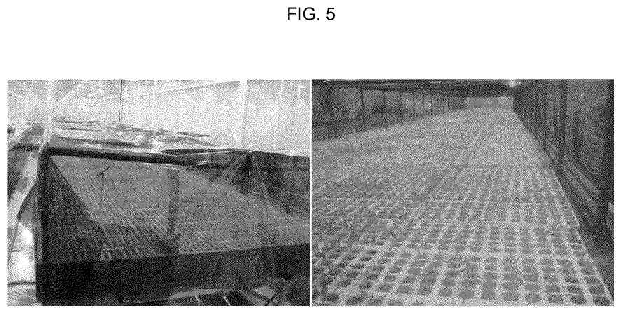

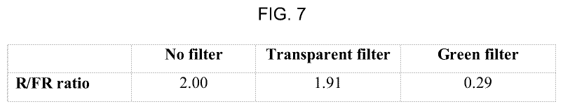

A high throughput screening method was developed for the identification of plants with reduced shade avoidance syndrome (SAS). SAS commonly occurs in indoor farming conditions (FIG. 1) and is known to be induced by low R/FR light conditions. Green filters can efficiently and uniformly reduce the R/FR ratio reaching the plants. Here, a commercially available green filter (Lee Filter Roll 122 Fern Green) was placed between the plants and the light source,. The green filter reduced the R:FR ratio more than 6-fold: the R/FR ratio was 0.29 under the green filter, compared to 1.91 under transparent filter and 2.00 when no filter was present; see FIG. 7 and FIG. 2; FIG. 3; FIG. 5). Under the green filter the WT lettuce plants showed all the physiologic al responses that are typical for the shade avoidance syndrome, namely elongated stem internodes, reduced leaf lamina area, slightly pale color of the leaves, elongated petioles and reduced root development. Our experimental setup had a throughput of approximately 17.000 young plants in a period of 2 weeks. The above-described screening method is suitable for all crops typically grown in indoor farming conditions.

### Example 2

### Quantitative Analysis of the Shade Avoidance Syndrome

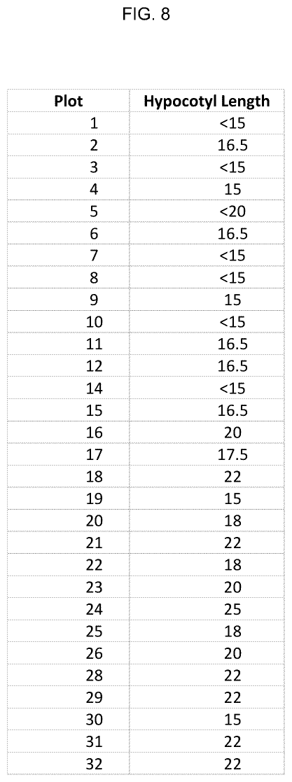

FIG. 8 shows the phenotypic analysis of shoot elongation responses, based on the length of the hypocotyl. Hypocotyl length of putative shade tolerant mutant plants were assessed in young seedling stage in plants grown for 26 days under selective conditions. Each plot contained 3 to 6 individual plants. Length is measured in mm, values represent the means of measurements done on individual plants within each plot. Plot 24 contains the data of the wild-type (Burovia) plants.

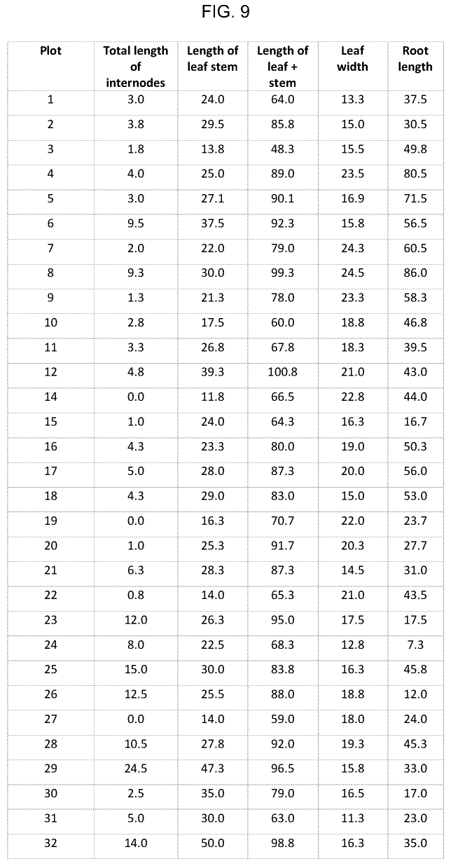

FIG. 9 shows the phenotypic analysis of shoot and root elongation responses. Root and shoot elongation responses of putative shade tolerant mutant plants were assessed in young seedling stage in plants grown under selective conditions. Each plot contained 3 to 6 individual plants. Length is measured in cm, values represent the means of measurements done on individual plants within each plot. Plot 24 contains the data of the wild-type (Burovia) plants.

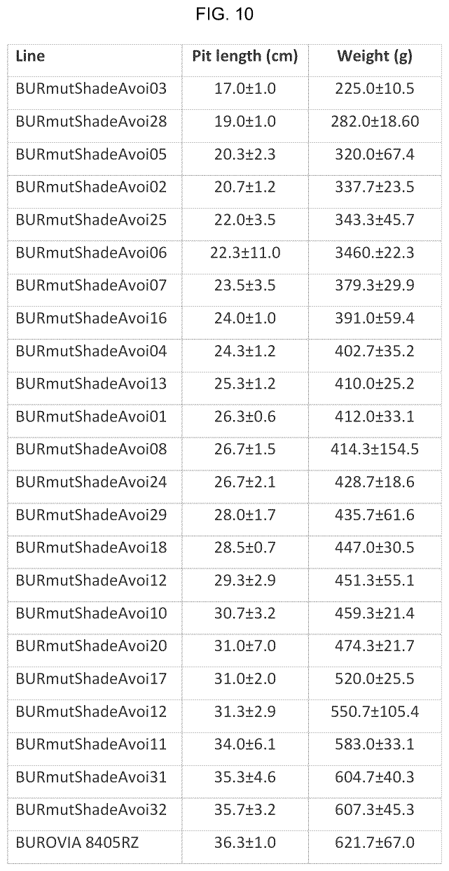

FIG. 10 shows the pit length (cm) and weight (g) of fully grown lettuce heads of selected shade tolerant mutant lines compared to the non-mutated parent (Burovia RZ). The mutant plants were grown in a greenhouse under average conditions of 16 h day time at 20° C. /8 h night time at 17° C. The values represent means of 3 individuals.

### Example 3

### Selection of Shade Tolerant Mutant Lines

### 3.1 Visual Selection of Divergent Phenotypes

Lettuce seeds were mutagenized in order to introduce mutations into the genome. Point mutations were introduced using EMS. The mutagenized seeds were then germinated and the resultant M1 plants were selfed or crossed to produce M2 seed. M2 seeds were sown in trays under green filter and grown for two to three weeks until the cotyledons were fully expanded and the first true leaves became visible (young seedling stage). At this stage plants were selected that did not show the shade avoidance phenotype and resembled a wild-type plant grown under normal conditions, in order to avoid the selection of (semi)dwarfing plants. The selected plants were grown to maturity and their seeds, obtained through selfing, were harvested (M3 seeds).

34,000 EMS-mutagenized M2 seeds from lettuce variety Burovia were sown under green filter. Three weeks after sowing, at young seedling stage, plants were screened for individuals that did not display the characteristic traits for shade avoidance. The selected plants did not have elongated hypocotyl and petiole, did not show petiole hyponasty, had dark green leaves and large leaf lamina. In this experiment about 40 shade avoidance mutants could be identified. These mutants were transplanted, grown to maturity and their seeds were collected.

3.2 Visual Selection of Lines with Homozygous Mutations (Progeny Testing)

The inbred M3 seeds obtained from the previous screening were sown in a further test under selective conditions, i.e. with green filter. The plants were selected as before, at young seedling stage, and assessed for the following characteristics: total length of internodes, length of leaf stem, plant length (stem and leaf combined), length of leaf lamina and root length. The characteristics are measured and compared to similar characteristics of the non-mutated cultivar they originated from, grown under identical conditions, in the same experiment. The best performing lines are selected, transplanted into individual pots, allowed to self-pollinate and their seeds are collected.

M3 seeds of 31 mutant lines were sown and tested under selective conditions. At young seedling stage the plants were assessed for their shade avoidance syndrome. The results of the measurements are given in FIG. 9.

### 3.3 Further Evaluation of Shade Tolerant Mutant Lines

General value traits were examined and the consistency of the short-internode phenotype was evaluated in greenhouse and hydroponic conditions, in order to verify the performance of the mutant lines in various commercial growing settings.

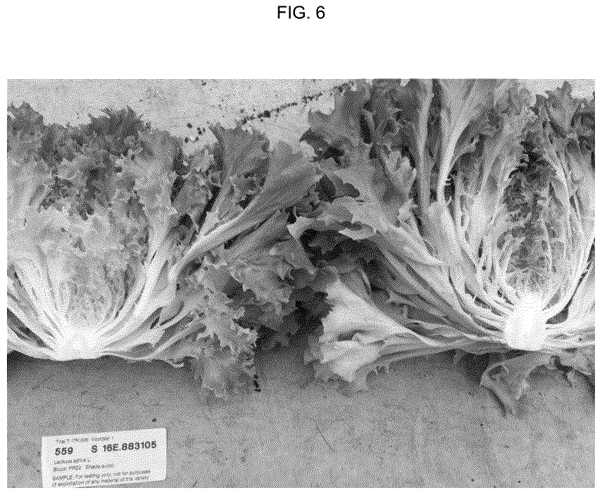

A set of mutant lines was grown under the greenhouse conditions as defined herein, to test the consistency of the shade tolerant phenotype. The mutant plants were harvested when fully grown, just before complete maturity, and assessed for weight and pit length (FIG. 10, FIG. 6). Furthermore, the mutant lines were also assessed for thermomorphogenesis response. This experiment revealed that a subset of the shade tolerant mutants also had a reduced thermomorphogenesis response. Based on the combined data of all experiments, three mutant lines were selected for genetic mapping and breeding purposes.

### Example 4

### Genetic Characterization of the Shade Tolerance Trait

The selected mutant lines were genetically characterized by mapping-by-sequencing, in order to identify the modified gene or genes responsible for the trait.

Three selected mutant lines were mapped (mut3, mut4, mut5). For mapping purposes a backcross segregating population was made for each line. Extreme phenotypes of these populations were bulked and their DNA was pooled and sequenced. The mapping-by-sequencing approach revealed several loci that were under selection.

Based on this information, new markers were developed for fine mapping. In the case of mut4 SNP mutations on linkage group 6 were found to correlate with the phenotype. Fine-mapping revealed that the causal gene is located between positions 198192198 and 203437218 on lg6.

Based on this result it was concluded that a SNP in gene EVM6.28510 is the polymorphism causing the mutant phenotype. The protein encoded by this gene is annotated as ent-kaurene oxidase-2 (E.C. 1.14.13.78). The SNP mutation CAG→TAG at position 200841726 on lg6 is a recessive mutation, resulting in a premature stop-codon at amino acid position 368, thus effectively truncating the encoded protein, which has a wild-type length of 512 amino acids. The truncated protein shows a loss-of-function protein. The predicted amino acid sequences for this protein in wild-type lettuce (variety Burovia) and in mut4 are given in SEQ ID NO.: 3 and SEQ ID NO.: 4, respectively. The nucleotide sequence for the marker specific for this SNP is listed in FIG. 11.

For mut3 and mut5, SNP mutations on lg9 were found to correlate with the phenotype and fine-mapping revealed that both mutants had originally been derived from the same mutation event. A SNP in gene EVM9.9794 appeared to be the cause for the mutant phenotype. The protein encoded by this gene is annotated as gibberellin-20-oxidase 1-B. The SNP mutation (GAA→AAA at position 72575669 on lg9) is recessive and results in a non-conservative amino acid change, replacing the negatively charged glutamine with a positively charged lysine (Glu→Lys), at position 268 of the encoded gibberellin-20-oxidase protein, which has a wild-type length of 383 amino acids. The predicted amino acid sequences for this protein in wild-type lettuce (variety Burovia) and in mut3 and mut5 are shown in SEQ ID NO.: 7 and SEQ ID NO.: 8, respectively; the mutated amino acid is shown in bold letter. The sequence for marker LS06187, specifically designed for the SNP in lg_9.9794, is given in FIG. 12.

The invention is further described by the following numbered paragraphs:

1. A lettuce plant comprising a modified LsKO2 gene and/or a modified Ls20ox1-B gene, wherein the homozygous presence of said genes leads to a shade tolerant phenotype,
wherein the wild-type LsKO2 gene comprises a coding sequence having at least 70% sequence identity to SEQ ID NO.: 9 and the wild-type Ls20ox1-B gene comprises a coding sequence having at least 70% sequence identity to SEQ ID NO.: 11,
wherein the modification comprises replacement and/or deletion and/or insertion of nucleotides resulting in an absence of functional KO2 and/or GA20ox1-B protein or wherein the modification results in the absence of the wild-type LsKO2 and/or Ls20ox1-B gene, and
wherein the homozygous presence of one or both of the modified LsKO2 and Ls20ox1-B genes in the plant or the homozygous absence of one or both of the wild-type LsKO2 and/or Ls20ox1-B genes from the plant confers shade tolerance to the plant as compared to a plant comprising the wild-type LsKO2 and Ls20ox1-B gene and not showing shade tolerance.

2. The lettuce plant of paragraph 1, wherein the lettuce plant is shade tolerant as a result of the homozygous presence of the modified LsKO2 gene.

3. The lettuce plant of paragraph 1, wherein the lettuce plant is shade tolerant as a result of the homozygous absence of the wild-type LsKO2 gene.

4. The lettuce plant of paragraph 1, wherein the modified LsKO2 gene comprises a premature stop codon.

5. The lettuce plant of paragraph 2, wherein the modified LsKO2 gene comprises a premature stop codon.

6. The lettuce plant of paragraph 4, wherein the premature stop codon is located within the part encoding the cytochrome p450 domain of the KO2 protein.

7. The lettuce plant of paragraph 5, wherein the premature stop codon is located within the part encoding the cytochrome p450 domain of the KO2 protein.

8. The lettuce plant of paragraph 4, wherein the modified LsKO2 gene encodes a truncated protein of 367 amino acids or less.

9. The lettuce plant of paragraph 5, wherein the modified LsKO2 gene encodes a truncated protein of 367 amino acids or less.

10. The lettuce plant of paragraph 1, wherein the stop codon is the result of a substitution of a cytosine by a thymine at position 1102 of SEQ ID NO.: 9.

11. The lettuce plant of paragraph 2, wherein the stop codon is the result of a substitution of a cytosine by a thymine at position 1102 of SEQ ID NO.: 9.

12. The lettuce plant of paragraph 1, wherein the modified LsKO2 gene is as comprised in the genome of seeds of which a representative sample is deposited under accession number NCIMB 43547.

13. The lettuce plant of paragraph 2, wherein the modified LsKO2 gene is as comprised in the genome of seeds of which a representative sample is deposited under accession number NCIMB 43547.

14. The lettuce plant of paragraph 1, wherein the lettuce plant is shade tolerant as a result of the homozygous presence of the modified Ls20ox1-B gene.

15. The lettuce plant of paragraph 1, wherein the lettuce plant is shade tolerant as a result of the homozygous absence of the wild-type Ls20ox1-B gene.

16. The lettuce plant of paragraph 1, wherein the modified Ls20ox1-B gene comprises a non-conservative amino acid replacement.

17. The lettuce plant of paragraph 14, wherein the modified Ls20ox1-B gene comprises a non-conservative amino acid replacement.

18. The lettuce plant of paragraph 16, wherein the non-conservative amino acid replacement is located within the part encoding the Fe2OG-dioxygenase domain of the GA20ox1-B protein.

19. The lettuce plant of paragraph 17, wherein the non-conservative amino acid replacement is located within the part encoding the Fe2OG-dioxygenase domain of the GA20ox1-B protein.

20. The lettuce plant of paragraph 16, wherein the modified gene encodes a non-functional protein.

21. The lettuce plant of paragraph 17, wherein the modified gene encodes a non-functional protein.

22. The lettuce plant of paragraph 18, wherein the modified gene encodes a non-functional protein.

23. The lettuce plant of paragraph 19, wherein the modified gene encodes a non-functional protein.

24. The lettuce plant of paragraph 16, wherein the non-conservative amino acid replacement is the result of a substitution of a guanine by an adenine at position 766 of SEQ ID NO.: 11.

25. The lettuce plant of paragraph 17, wherein the modified gene encodes a non-functional protein.

26. The lettuce plant of paragraph 1, wherein the modified Ls20ox1-B gene is as comprised in the genome of seeds of which a representative sample is deposited under accession number NCIMB 43548.

27. The lettuce plant of paragraph 14, wherein the modified Ls20ox1-B gene is as comprised in the genome of seeds of which a representative sample is deposited under accession number NCIMB 43548.

28. A lettuce seed comprising the modified LsKO2 gene and/or the modified Ls20ox1-B gene of paragraph 1, wherein the plant grown from said seed shows shade tolerance as a result of the homozygous presence of the modified LsKO2 gene and/or the modified Ls20ox1-B gene.

29. A propagation material capable of developing into and/or being derived from the plant of paragraph 1, wherein the propagation material is suitable for sexual reproduction, and is in particular selected from a microspore, pollen, ovary, ovule, embryo sac and egg cell, or is suitable for vegetative reproduction, and is in particular selected from a cutting, root, stem cell, and protoplast, or is suitable for tissue culture of regenerable cells or protoplasts, which regenerable cells or protoplasts are in particular selected from a leaf, pollen, embryo, cotyledon, hypocotyl, meristematic cell, root, root tip, anther, flower and stem.

30. The modified LsKO2 gene of paragraph 1, wherein the LsKO2 gene is modified compared to the wild-type according to SEQ ID NO.: 1, and wherein the modified LsKO2 gene confers shade tolerance when present homozygously.

31. The modified LsKO2 gene of paragraph 30, wherein the modification in the LsKO2 gene results in a protein showing a loss-of-function or reduction-of-function or in the absence of a protein expression product.

32. The modified LsKO2 gene of paragraph 31, wherein the loss-of-function or reduction-of-function is the result of a premature stop codon.

33. The modified LsKO2 gene of paragraph 32, wherein the premature stop codon is caused by a single nucleotide substitution of a C with a T at nucleotide position 1102 of SEQ ID NO.: 9.

34. The modified Ls20ox1-B gene of paragraph 1, wherein the Ls20ox1-B gene is modified compared to the wild-type according to SEQ ID NO.: 5, and wherein the modified Ls20ox1-B gene confers shade tolerance when present homozygously.

35. The modified Ls20ox1-B gene of paragraph 34, wherein the modification in the Ls20ox1-B gene results in a protein showing loss-of-function or reduction-of-function or in the absence of protein expression product.

36. The modified Ls20ox1-B gene of paragraph 35, wherein the loss-of-function or reduction-of-function is the result of a nucleotide substitution.

37. The modified Ls20ox1-B gene of paragraph 36, wherein the modified Ls20ox1-B gene comprises a substitution of a G with an A at position 766 of SEQ ID NO.: 11.

38. A marker that identifies the modified LsKO2 gene of paragraph 1, wherein the marker detects a substitution from a cytosine to a thymine at position 1102 of the wild-type LsKO2 gene sequence of SEQ ID NO.: 9, or wherein the marker detects said substitution on a corresponding position of a homologous sequence that has at least 70% sequence identity to SEQ ID NO.: 9.

39. A marker that identifies the modified Ls20ox1-B gene of paragraph 1, wherein the marker detects a substitution from a guanine to an adenine at position 766 of the wild-type Ls20ox1-B gene sequence of SEQ ID NO.: 11, or wherein the marker detects said substitution on a corresponding position of a homologous sequence that has at least 70% sequence identity to SEQ ID NO.: 11.

40. The marker of paragraph 38, the sequence of which is as provided in FIG. 11 and FIG. 12, for use in the identification or development of a shade tolerant lettuce plant, or for use in the development of other markers linked to a modified LsKO2 and/or modified Ls20ox1-B homolog.

41. The marker of paragraph 39, the sequence of which is as provided in FIG. 11 and FIG. 12, for use in the identification or development of a shade tolerant lettuce plant, or for use in the development of other markers linked to a modified LsKO2 and/or modified Ls20ox1-B homolog.

42. A method for identifying a shade tolerant lettuce plant, wherein the method comprises screening a plant or a population of plants for the presence of the modified LsKO2 gene and/or Ls20ox1-B gene of paragraph 1, and identifying the plant which homozygously comprises the modification in one or both genes, optionally followed by a phenotypic screening, as a plant showing shade tolerance.

43. The method of paragraph 42, further comprising the step of selecting the shade tolerant plant.

44. A method for producing a shade tolerant plant, comprising the step of introducing a mutation in the LsKO2 gene and/or Ls20ox1-B gene by random or site-directed mutagenesis, wherein the mutation results in an absence of functional KO2 protein and/or GA20ox1-B protein in said plant.

45. A method for producing a shade tolerant plant, said method comprising;
a) crossing a plant comprising a modified LsKO2 and/or Ls20ox1-B gene of paragraph 1, with another plant to obtain an F1 population;
b) optionally performing one or more rounds of selfing and/or crossing a plant from the F1 to obtain a further generation population;
c) selecting from the population a plant that comprises the modified LsKO2 and/or Ls20ox1-B gene and is shade tolerant.

46. A method of producing a hybrid plant seed comprising crossing a first parent plant with a second parent plant and harvesting the resultant plant seed, wherein said first parent plant and/or said second parent plant comprises either one of the modified LsKO2 and/or Ls20ox1-B gene of paragraph 1.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A lettuce plant comprising a modified LsKO2 gene and/or a modified Ls20ox1-B gene, wherein the homozygous presence of said genes leads to a shade tolerant phenotype,
wherein the wild-type LsKO2 gene comprises a coding sequence having at least 70% sequence identity to SEQ ID NO.: 9 and the wild-type Ls20ox1-B gene comprises a coding sequence having at least 70% sequence identity to SEQ ID NO.: 11,
wherein the modification comprises replacement and/or deletion and/or insertion of nucleotides resulting in an absence of functional KO2 and/or GA20ox1-B protein or wherein the modification results in the absence of the wild-type LsKO2 and/or Ls20ox1-B gene, and
wherein the homozygous presence of one or both of the modified LsKO2 and Ls20ox1-B genes in the plant or the homozygous absence of one or both of the wild-type LsKO2 and/or Ls20ox1-B genes from the plant confers shade tolerance to the plant as compared to a plant comprising the wild-type LsKO2 and Ls20ox1-B gene and not showing shade tolerance.

2. The lettuce plant of claim 1, wherein the lettuce plant is shade tolerant as a result of the homozygous presence of the modified LsKO2 gene.

3. The lettuce plant of claim 1, wherein the lettuce plant is shade tolerant as a result of the homozygous absence of the wild-type LsKO2 gene.

4. The lettuce plant of claim 1, wherein the modified LsKO2 gene comprises a premature stop codon.

5. The lettuce plant of claim 2, wherein the modified LsKO2 gene comprises a premature stop codon.

6. The lettuce plant of claim 4, wherein the premature stop codon is located within the part encoding the cytochrome p450 domain of the KO2 protein.

7. The lettuce plant of claim 5, wherein the premature stop codon is located within the part encoding the cytochrome p450 domain of the KO2 protein.

8. The lettuce plant of claim 4, wherein the modified LsKO2 gene encodes a truncated protein of 367 amino acids or less.

9. The lettuce plant of claim 5, wherein the modified LsKO2 gene encodes a truncated protein of 367 amino acids or less.

10. The lettuce plant of claim 1, wherein the stop codon is the result of a substitution of a cytosine by a thymine at position 1102 of SEQ ID NO.: 9.

11. The lettuce plant of claim 2, wherein the stop codon is the result of a substitution of a cytosine by a thymine at position 1102 of SEQ ID NO.: 9.

12. The lettuce plant of claim 1, wherein the modified LsKO2 gene is as comprised in the genome of seeds of which a representative sample is deposited under accession number NCIMB 43547.

13. The lettuce plant of claim 2, wherein the modified LsKO2 gene is as comprised in the genome of seeds of which a representative sample is deposited under accession number NCIMB 43547.

14. The lettuce plant of claim 1, wherein the lettuce plant is shade tolerant as a result of the homozygous presence of the modified Ls20ox1-B gene.

15. The lettuce plant of claim 1, wherein the lettuce plant is shade tolerant as a result of the homozygous absence of the wild-type Ls20ox1-B gene.

16. The lettuce plant of claim 1, wherein the modified Ls20ox1-B gene comprises a non-conservative amino acid replacement.

17. The lettuce plant of claim 14, wherein the modified Ls20ox1-B gene comprises a non-conservative amino acid replacement.

18. The lettuce plant of claim 16, wherein the non-conservative amino acid replacement is located within the part encoding the Fe2OG-dioxygenase domain of the GA20ox1-B protein.

19. The lettuce plant of claim 17, wherein the non-conservative amino acid replacement is located within the part encoding the Fe2OG-dioxygenase domain of the GA20ox1-B protein.

20. The lettuce plant of claim 16, wherein the modified gene encodes a non-functional protein.

21. The lettuce plant of claim 17, wherein the modified gene encodes a non-functional protein.

22. The lettuce plant of claim 18, wherein the modified gene encodes a non-functional protein.

23. The lettuce plant of claim 19, wherein the modified gene encodes a non-functional protein.

24. The lettuce plant of claim 16, wherein the non-conservative amino acid replacement is the result of a substitution of a guanine by an adenine at position 766 of SEQ ID NO.: 11.

25. The lettuce plant of claim 17, wherein the modified gene encodes a non-functional protein.

26. The lettuce plant of claim 1, wherein the modified Ls20ox1-B gene is as comprised in the genome of seeds of which a representative sample is deposited under accession number NCIMB 43548.

27. The lettuce plant of claim 14, wherein the modified Ls20ox1-B gene is as comprised in the genome of seeds of which a representative sample is deposited under accession number NCIMB 43548.

28. A lettuce seed comprising the modified LsKO2 gene and/or the modified Ls20ox1-B gene of claim 1, wherein the plant grown from said seed shows shade tolerance as a result of the homozygous presence of the modified LsKO2 gene and/or the modified Ls20ox1-B gene.

29. A propagation material capable of developing into and/or being derived from the plant of claim 1, wherein the propagation material is suitable for sexual reproduction, and is in particular selected from a microspore, pollen, ovary, ovule, embryo sac and egg cell, or is suitable for vegetative reproduction, and is in particular selected from a cutting, root, stem cell, and protoplast, or is suitable for tissue culture of regenerable cells or protoplasts, which regenerable cells or protoplasts are in particular selected from a leaf, pollen, embryo, cotyledon, hypocotyl, meristematic cell, root, root tip, anther, flower and stem.

30. The modified LsKO2 gene of claim 1, wherein the LsKO2 gene is modified compared to the wild-type according to SEQ ID NO.: 1, and wherein the modified LsKO2 gene confers shade tolerance when present homozygously.

31. The modified LsKO2 gene of claim 30, wherein the modification in the LsKO2 gene results in a protein showing a loss-of-function or reduction-of-function or in the absence of a protein expression product.

32. The modified LsKO2 gene of claim 31, wherein the loss-of-function or reduction-of-function is the result of a premature stop codon.

33. The modified LsKO2 gene of claim 32, wherein the premature stop codon is caused by a single nucleotide substitution of a C with a T at nucleotide position 1102 of SEQ ID NO.: 9.

34. The modified Ls20ox1-B gene of claim 1, wherein the Ls20ox1-B gene is modified compared to the wild-type according to SEQ ID NO.: 5, and wherein the modified Ls20ox1-B gene confers shade tolerance when present homozygously.

35. The modified Ls20ox1-B gene of claim 34, wherein the modification in the Ls20ox1-B gene results in a protein showing loss-of-function or reduction-of-function or in the absence of protein expression product.

36. The modified Ls20ox1-B gene of claim 35, wherein the loss-of-function or reduction-of-function is the result of a nucleotide substitution.

37. The modified Ls20ox1-B gene of claim 36, wherein the modified Ls20ox1-B gene comprises a substitution of a G with an A at position 766 of SEQ ID NO.: 11.

38. A marker that identifies the modified LsKO2 gene of claim 1, wherein the marker detects a substitution from a cytosine to a thymine at position 1102 of the wild-type LsKO2 gene sequence of SEQ ID NO.: 9, or wherein the marker detects said substitution on a corresponding position of a homologous sequence that has at least 70% sequence identity to SEQ ID NO.: 9.

39. A marker that identifies the modified Ls20ox1-B gene of claim 1, wherein the marker detects a substitution from a guanine to an adenine at position 766 of the wild-type Ls20ox1-B gene sequence of SEQ ID NO.: 11, or wherein the marker detects said substitution on a corresponding position of a homologous sequence that has at least 70% sequence identity to SEQ ID NO.: 11.

40. The marker of claim 38, the sequence of which is as provided in FIG. 11 and FIG. 12, for use in the identification or development of a shade tolerant lettuce plant, or for use in the development of other markers linked to a modified LsKO2 and/or modified Ls20ox1-B homolog.

41. The marker of claim 39, the sequence of which is as provided in FIG. 11 and FIG. 12, for use in the identification or development of a shade tolerant lettuce plant, or for use in the development of other markers linked to a modified LsKO2 and/or modified Ls20ox1-B homolog.

42. A method for identifying a shade tolerant lettuce plant, wherein the method comprises screening a plant or a population of plants for the presence of the modified LsKO2 gene and/or Ls20ox1-B gene of claim 1, and identifying the plant which homozygously comprises the modification in one or both genes, optionally followed by a phenotypic screening, as a plant showing shade tolerance.

43. The method of claim 42, further comprising the step of selecting the shade tolerant plant.

44. A method for producing a shade tolerant plant, comprising the step of introducing a mutation in the LsKO2 gene and/or Ls20ox1-B gene by random or site-directed mutagenesis, wherein the mutation results in an absence of functional KO2 protein and/or GA20ox1-B protein in said plant.

45. A method for producing a shade tolerant plant, said method comprising;
a) crossing a plant comprising a modified LsKO2 and/or Ls20ox1-B gene of claim 1, with another plant to obtain an F1 population;
b) optionally performing one or more rounds of selfing and/or crossing a plant from the F1 to obtain a further generation population;
c) selecting from the population a plant that comprises the modified LsKO2 and/or Ls20ox1-B gene and is shade tolerant.

46. A method of producing a hybrid plant seed comprising crossing a first parent plant with a second parent plant and harvesting the resultant plant seed, wherein said first parent plant and/or said second parent plant comprises either one of the modified LsKO2 and/or Ls20ox1-B gene of claim 1.
